(19) United States
(12) Patent Application Publication
(10) Pub. No.: US 2003/0087956 A1
(43) Pub. Date: May 8, 2003

(54) Urea compounds as inhibitors for vla-4

(76) Inventors: Craig Johnstone, Macclesfield, Cheshire (GB); Michael Stewart Large, Macclesfield Cheshire (GB)

Correspondence Address: ROPES & GRAY, ONE INTERNATIONAL PLACE, BOSTON, MA, 02110-2624, US

(21) Appl. No.: 10/149,932

(22) PCT Filed: Jan. 17, 2001
(86) PCT No.: PCT/GB01/00162

(30) Foreign Application Priority Data
Jan. 21, 2000 (GB) 0001348.2

(51) Int. Cl. A61K 31/36; A61K 31/277; A61K 31/195; A61K 31/24; A61K 31/165

(52) U.S. Cl. 514/464; 514/561; 514/563; 514/521; 514/562; 514/619; 514/620; 549/434; 558/418; 558/419; 562/426; 562/455

(57) ABSTRACT

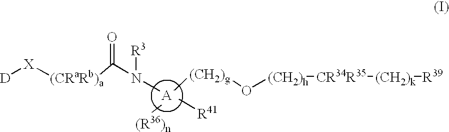

A compound of formula (I) where D is a VLA-4 specificity determinant which does not impart significant IIB/IIIa activity; R⁴¹ is a group of the formula (V): U - (CH₂)_{d} - V - T wherein U is selected from oxygen, sulphur, a direct bond or —CH₂O—, V is selected from nitrogen, oxygen, sulphur, S(O), S(O)₂ or a direct bond, d is zero or a number from 1 to 4, and T is selected from a range of variables defined in the application; and other variables are defined in the application, or a pharmaceutically acceptable salt or in vivo hydrolysable derivative thereof. The compounds are useful in the treatment of disease mediated by the interaction between VCAM-1 and/or fibronectin and the integrin receptor α₄β₁. Pharmaceutical compositions and methods of use or treatment are also described and claimed.

This invention relates to compounds which are inhibitors of the interaction between the integrin α₄β₁, also known as Very Late Antigen-4 (VLA-4) or CD49d/CD29, and its protein ligands, for example Vascular Cell Adhesion Molecule-1 (VCAM-1) and fibronectin. This invention further relates to processes for preparing such compounds, to pharmaceutical compositions containing them and to their use in methods of therapeutic application.

α₄β₁ is a member of the integrin family of heterodimeric cell surface receptors that are composed of noncovalently associated glycoprotein subunits (α and β) and are involved in cell adhesion to other cells or to extracellular matrix. There are at least 14 different human integrin α subunits and at least 8 different β subunits and each β subunit can form a heterodimer with one or more α subunits. Integrins can be subdivided based on their β subunit composition. α₄β₁ is one of several β₁ integrins, also known as Very Late Antigens (VLA).

The interactions between integrins and their protein ligands are fundamental for maintaining cell function, for example by tethering cells at a particular location, facilitating cell migration, or providing survival signals to cells from their environment. Ligands recognised by integrins include extracellular matrix proteins, such as collagen and fibronectin; plasma proteins, such as fibrinogen; and cell surface molecules, such as transmembrane proteins of the immunoglobulin superfamily and cell-bound complement. The specificity of the interaction between integrin and ligand is governed by the α and β subunit composition.

Integrin α₄β₁ is expressed on numerous hematopoietic cells and established cell lines, including hematopoietic precursors, peripheral and cytotoxic T lymphocytes, B lymphocytes, monocytes, thymocytes and eosinophils [Hemler, M. E. et al (1987), J. Biol. Chem., 262, 11478-11485; Bochner, B. S. et al (1991), J. Exp. Med., 173, 1553-1556]. Unlike other β₁ integrins that bind only to cell-extracellular matrix proteins, α₄β₁ binds to VCAM-1, an immunoglobulin superfamily member expressed on the cell surface, for example on vascular endothelial cells, and to fibronectin containing the alternatively spliced type III connecting segment (CS-1 fibronectin) [Elices, M. J. et al (1990), Cell, 60, 577-584; Wayner, E. A. et al (1989). J. Cell Biol., 109, 1321-1330].

The activation and extravasation of blood leukocytes plays a major role in the development and progression of inflammatory diseases. Cell adhesion to the vascular endothelium is required before cells migrate from the blood into inflamed tissue and is mediated by specific interactions between cell adhesion molecules on the surface of vascular endothelial cells and circulating leukocytes [Sharar, S. R. et al (1995). Springer Semin. Immunopathol, 16, 359-378]. α₄β₁ is believed to have an important role in the recruitment of lymphocytes, monocytes and eosinophils during inflammation. α₄β₁/ligand binding has also been implicated in T-cell proliferation, B-cell localisation to germinal centres, haemopoeitic progenitor cell localisation in the bone marrow, placental development, muscle development and tumour cell metastasis.

The affinity of α₄β₁ for its ligands is normally low but chemokines expressed by inflamed vascular endothelium act via receptors on the leukocyte surface to upregulate α₄β₁ fuction [Weber, C. et al (1996), J. Cell Biol., 134, 1063-1073]. VCAM-1 expression is upregulated on endothelial cells in vitro by inflammatory cytokines [Osborn, L. et al (1989) Cell, 59, 1203-1211] and in human inflammatory diseases such as rheumatoid arthritis [Morales-Ducret, J. et al (1992). J. Immunol., 149, 1424-1431], multiple sclerosis [Cannella, B. et al., (1995). Ann Neurol., 37, 424-435], allergic asthma [Fukuda, T. et al (1996), Am. J. Respir. Cell Mol. Biol., 14, 84-94] and atherosclerosis [O'Brien, K. D. et al (1993). J. Clin. Invest, 92, 945-951].

Monoclonal antibodies directed against the α₄ integrin subunit have been shown to be effective in a number of animal models of human inflammatory diseases including multiple sclerosis, rheumatoid arthritis, allergic asthma, contact dermatitis, transplant rejection, insulin-dependent diabetes, inflammatory bowel disease, and glomerulonephritis.

Integrins recognise short peptide motifs in their ligands The minimal α₄β₁ binding epitope in CS-1 is the tripeptide leucine-aspartic acid-valine (Leu-Asp-Val) [Komoriya, A., et al (1991). J. Biol. Chem, 266, 15075-15079] while VCAM-1 contains the similar sequence isoleucine-aspartic acid-serine [Clements, J. M., et al (1994). J. Cell Sci., 107, 2127-2135]. The 25-amino acid fibronectin fragment, CS-1 peptide, which contains the Leu Asp-Val motif, is a competitive inhibitor of α₄β₁ binding to VCAM-1 [Makarem, R., et al (1994). J. Biol. Chem., 269, 4005-4011]. Small molecule α₄β₁ inhibitors based on the Leu-Asp-Val sequence in CS-1 have been described, for example the linear molecule phenylacetic acid-Leu-Asp-Phe-D-Pro-amide [Molossi, S. et al (1995). J. Clin. Invest, 95, 2601-2610] and the disulphide cyclic peptide Cys-Trp-Leu-Asp-Val-Cys [Vanderslice, P., et al (1997). J. Immunol., 158, 1710-1718].

More recently, non- and semi-peptidic compounds which inhibit α₄β₁/VCAM binding and which can be orally administered have been reported in for example, WO96/22966 and WO98/04247.

There remains a continuing need for alternative compounds which inhibit the interaction between VCAM-1 and fibronectin with integrin α₄β₁ and, in particular, for compounds which can be administered by an oral route.

We have now found a group of compounds which contain a substituted ring system which inhibit this interaction.

Accordingly the present invention provides a compound of formula (I)

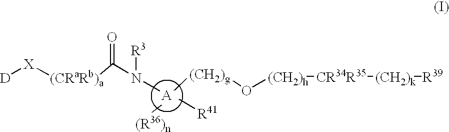

where D is a VLA-4 specificity determinant which does not impart significant IIB/IIIa activity;

R^{a} and R^{b} are independently hydrogen or C₁₋₄ alkyl;

a is an integer from 1 to 4;

X is a direct bond, oxygen, sulphur, amino or C₁₋₄alkylammno;

R³ is hydrogen or C₁₋₅ alkyl;

A is aryl or heterocycle;

n is 0 or an integer of from 1 to 3;

R³⁴ is hydrogen, C₁₋₆ alkyl, aryl or heterocycle, the aryl or heterocycle being optionally substituted with one or more substituents independently selected fiom nitro, C₁₋₆alkyl, C₂₋₆alkenyl, C₂₋₆alkynyl, C₁₋₄alkoxy, C₁₋₆alkylamino, C₁₋₄alkylC₁₋₆alkyoxyl, C₁₋₆alkylaminoC₁₋₆alkyl, cyano, halogeno, trifluoromethyl, hydroxy, (CH₂)ₚOH where p is 1 or 2, —CO₂R^{e1} and —CONR^{e1}R^{f1}, where R^{e1} and R^{f1} are independently selected from hydrogen and C₁₋₆ alkyl;

R³⁵ is selected from hydrogen, hydroxy, C₁₋₆ alkyl, C₂₋₆alkenyl, 1,3-benzodioxol-5-yl, an ester group, amido, heterocycle and aryl, the heterocycle, and aryl optionally substituted with one or more substituents independently selected from nitro, C₁₋₆alkyl, C₂₋₆alkenyl, C₂₋₆alkynyl, C₁₋₄alkoxy, C₁₋₆alkylamino, C₁₋₄alkylC₁₋₆alkyoxyl, C₁₋₆alkylaminoC₁₋₆alkyl, cyano, halogeno, trifluoromethyl, hydroxy, (CH₂)ₚOH where p is 1 or 2, —CO₂R^{e2} and —CONR^{e2}R^{f2}, where R^{e2} and R^{f2} are independently selected from hydrogen and C₁₋₆ alkyl;

each R³⁶ group, which may be the same or different, is independently selected from C₁₋₆alkyl, C₂₋₆alkenyl, C₂₋₆alkynyl, C₁₋₄alkoxy, C₁₋₄alkanoyl, C₁₋₆alkylamino, C₁₋₄alkoxylC₁₋₆alkyl, C₁₋₆alkylaminoC₁₋₆alkyl, nitro, cyano, halogeno, trifluoromethyl, hydroxy, (CH₂)ₚOH where p is 1 or 2, —CO₂R^{e3}, and —CONR^{e3}R^{f3}, where R^{e3} and R^{f3} are independently selected from hydrogen and C₁₋₆ alkyl;

R³⁹ is an acidic functional group;

h is zero or 1;

g is zero of 1;

k is zero or a number from 1 to 3;

and R⁴¹ is a group of formula (V)

U - (CH₂)_{d} - V -T (V)

wherein U is selected from oxygen, sulphur, a direct bond or —CH₂O—, V is selected from nitrogen, oxygen, sulphur, S(O), S(O)₂ or a direct bond, d is zero or a number from 1 to 4, and T is selected from R^{c} or, when V is nitrogen, R^{c}R^{d}, where R^{c} and R^{d} are independently selected from hydrogen, C₁₋₄ alkyl, C₁₋₄ alkoxy, C₁₋₄ alkoxy(C₁₋₆)alkyl, C₃₋₇ cycloalkyl, aralkyl or aryl; or T is a heterocycle containing up to three heteroatoms selected from nitrogen, oxygen and sulphur, optionally substituted with one or more substituents selected from C₁₋₆alkyl, C₂₋₆alkenyl, C₂₋₆alkynyl, C₁₋₆ alkoxy, C₁₋₄alkanoyl, C₁₋₆ alkylamino, C₁₋₄alkoxylC₁₋₆alkyl, C₁₋₆alkylaminoC₁₋₆alkyl, C₁₋₄ alkylsulphonyl, nitro, cyano, halogeno, trifluoromethyl, trifluoromethoxy, hydroxy, (CH₂)ₚOH where p is 1 or 2, —CO₂R^{e4}, and —CONR^{e4}R^{f4}, where R^{e4} and R^{f4} are independently selected from hydrogen and C₁₋₆alkyl, and linked to V through a ring carbon or nitrogen and with the provisos that when T is a heterocycle linked to V through a ring nitrogen then V is a direct bond, and that at least one of U or V is other than a direct bond or d is othr than 0;

or a pharmaceutically acceptable salt or in vivo hydrolysable derivative thereof.

In this specification the following definitions are adopted:

The term ‘heterocycle’ means an aromatic or non-aromatic saturated or partially saturated cyclic ring system containing up to five heteroatoms independently selected from nitrogen, oxygen and sulphur. Heterocycles with two or more rings may include a mixture of aromatic and non-aromatic rings, or they may be completely aromatic or completely non-aromatic. Suitable optional substituents for heterocycles include one or more substituents selected from oxo, C₁₋₆alkyl, C₂₋₆alkenyl, C₂₋₆alkynyl, C₁₋₆alkoxy, C₁₋₄ alkanoyl, C₁₋₆alkylamino, C₁₋₄alkoxylC₁₋₆alkyl, C₁₋₆alkylaminoC₁₋₆alkyl, C₁₋₄ alkylsulphonyl, nitro, cyano, halogeno, trifluoromethyl, trifluoromethoxy, hydroxy, (CH₂)ₚOH where p is 1 or 2, —CO₂R^{e}, and —CONR^{e}R^{f}, where R^{e} and R^{f} are independently selected from hydrogen and C₁₋₆alkyl. Examples include 3 to 10 membered monocyclic or bicyclic rings with up to five heteroatoms selected from oxygen, nitrogen and sulphur, such as, for example, furanyl pyrrolinyl, piperidinyl, piperazinyl, thienyl, pyridyl, imidazolyl, tetrazolyl, thiazolyl, pyrazolyl, pyrimidinyl, triazinyl, pyridazinyl, pyrazinyl, morpholinyl, oxiranyl, oxetanyl, tetrahydrofuranyl, pyrrolidinyl, imidazolinyl, imidazolidinyl, pyrazolinyl, pyrazolidinyl, piperidinyl, homopiperazinyl, dihydropyridinyl, tetrahydropyridinyl, dihydropyrmidinyl and tetrahydropyrimidinyl. The monocyclic heteroaryl is a aromatic ring system containing up to four heteroatoms, examples of which are given above.

‘Bicyclic heteroaryl’ means an aromatic 5,6-6,5- or 6,6 fused ring system wherein one or both rings contain ring heteroatoms. The ring system may contain up to three heteroatoms, independently selected from oxygen, nitrogen or sulphur and can be optionally substituted with one or more substituents selected from C₁₋₆alkyl, C₂₋₆alkenyl, C₂₋₆alkynyl, C₁₋₆alkoxy, C₁₋₄alkanoyl, C₁₋₆alkylamino, C₁₋₄alkoxylC₁₋₆alkyl, C₁₋₆alkylaminoC₁₋₆alkyl, C₁₋₄ alkylsulphonyl, nitro, cyano, halogeno, trifluoromethyl, trifluoromethoxy, hydroxy, (CH₂)ₚOH where p is 1 or 2, —CO₂R^{e}, and —CONR^{e}R^{f}, where R^{e} and R^{f} are independently selected from hydrogen and C₁₋₆alkyl. When the ring system contains more than one heteratom at least one heteroatom is nitrogen. Examples of bicyclic heteroaryl's include quinazolinyl, benzothiophenyl, benzoxazolyl, benzimidazolyl, benzothiazolyl, benzofuranyl, indolyl, quinolinyl, phthalazinyl and benzotriazolyl.

‘Alkyl’ refers to straight or branched chain alkyl groups, which, unless otherwise stated, generally contain up to 10 suitably from 1-6 and more preferably from 1-4 carbon atoms. Similarly, the terms ‘alkenyl’ and ‘alkynyl’ refer to straight or branched unsaturated chains, which, unless otherwise stated, generally contain from 2-10 and preferably from 2-6 carbon atoms.

‘Aryl’ typically means phenyl or naphthyl, preferably phenyl.

‘Aralkyl’ means alkyl substituted by aryl such as benzyl.

The term ‘acidic functional group’ means a group which incorporates an acidic hydrogen and includes carboxylic acids, tetrazoles, acyl sulphonamides, sulphonic and sulphinic acids, and preferably is carboxy.

The term ‘ester group’ is an ester derived from a C₁₋₁₀ straight or branched alkyl arylalkyl or C₅₋₇ cycloalkyl (optionally substituted with C₁₋₄ allyl) alcohol. Suitable ester groups are those of formula —COOR″ where R″ can be tert-butyl, 2,4dimethyl-pent-3-yl, 4methyl-tetrahydropyranyl 2,2dimethyl aminoethyl or 2-methyl 3-phenyl prop-2-yl.

In this specification suitable specific groups for the substituents mentioned include:-

| | |
|---|---|
| for halogeno: | fluoro, chloro, bromo and iodo |
| for C₁₋₆alkyl | (this includes straight chained, branched |
| | structures and ring systems): methyl, |
| | ethyl, propyl, isopropyl, tert-butyl, |
| and | cyclopropane cyclohexane; |
| for C₂₋₆alkenyl: | vinyl, allyl and but-2-enyl; |
| for C₁₋₆alkanoyl; | formyl, acetyl, propionyl or butyryl; |
| for C₂₋₆alkynyl: | ethynyl, 2-propynyl and but-2-ynyl; |
| for C₁₋₆alkoxy: | ethoxy, ethoxy, propoxy, isopropoxy and |
| | butoxy; |
| for C₂₋₆alkenyloxy: | vinyloxy and allyloxy; |
| for C₂₋₆alkynyloxy: | ethynyloxy and 2-propynyloxy; |
| for C₁₋₆alkylamino: | methylamino, ethylamino, propylamino, |
| | isopropylamino and butylamino; |
| for di-C₁₋₆alkylamino: | dimethylamino, diethylamino; |
| for C₂₋₆alkanoylamino: | acetamido, propionamido and butyramido; |
| for N-C₁₋₆alkylcarbamoyl: | N-methylcarbamoyl, N-ethylcarbamoyl |
| | and N-propylcarbamoyl; |
| for C₁₋₆alkoxycarbonyl: | methoxycarbonyl, ethoxycarbonyl, |
| | propoxycarbonyl and tert-butoxycarbonyl; |
| for C₁₋₄alkoxyC₁₋₆alkyl: | methoxymethyl, ethoxymethyl, |
| | 1-methoxymethyl, 2-methoxyethyl; |
| for C₁₋₆alkylthio: | methylthio; |
| for C₁₋₄alkylsulphonyl: | methylsulphonyl; |
| for C₁₋₆alkylaminoC₁₋₆alkyl: | —CH₂NHC₂H₅ |

It is to be understood that, insofar as certain of the compounds of the formula (I) and (II) as defined above and below may exist in optically active or racernic forms by virtue of one or more asymmetric carbon atoms, the invention encompasses any such optically active or racemic form which can inhi bit the interaction between VCAM-1 and fibronectin with the integrin α₄β₁. The synthesis of optically active forms may be carried out by standard techniques of organic chemistry well known in the art, for example by synthesis from optically active staring materials or by resolution of a racemic form.

Suitably, in the compounds of formula (I), the group of sub-fonrula (i)

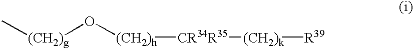

is arranged on the ring A at a position which is meta to the group of sub-formula (ii)

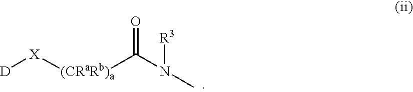

In a particular embodiment A is a 5 or 6-membered heterocycle or phenyl, and is preferably phenyl. Suitably, R⁴¹ is para to the group of sub-formula (i) and ortho to the group of sub-formula (ii) on the ring A.

Examples of suitable groups D are described in WO 98/042476. In particular, D is selected from the group consisting of alkyl aliphatic acyl, optionally substituted with N-alkyl- or N-arylamido, aroyl, heterocycloyl, alkyl- and arylsulfonyl, aralkylcarbonyl optionally substituted with aryl, heterocycloalkylcarbonyl, alkoxycarbonyl, aralkyloxycarbonyl, cycloalkylcarbonyl optionally fused with aryl, heterocycloalkoxycarbonyl, alkylaminocarbonyl, arylaminocarbonyl and aralkylaminocarbonyl optionally substituted with bis-(alkylsulfonyl)amino, alkoxycarbonylamino or alkenyl.

More preferably, D is selected from the group consistig of aliphatic acyl, aroyl, aralrylcarbonyl, heterocycloyl, alkoxycarbonyl, aralkyloxycarbonyl and heterocycloalkylcarbonyl, which may be optionally substituted as defined below. In other embodiments, D is preferably selected from the group consisting of (N-Ar′-urea)-para-substituted aralkylcarbonyl, (N-Ar′-urea)-para-substituted aralkyl and (N-Ar′-urea)-para-substituted aryl, where Ar′ or aryl groups may be optionally substituted as defined below. Most prefeably, A is selected from the group consisting of (N-Ar′-urea)-para-substituted phenylmethylcarbonyl, (N-Ar′-urea)-para-substituted phenylmethyl and (N-Ar′-urea)-para-substituted phenyl; where Ar′ is aryl such as phenyl. Any aryl group or Ar′ group present in D may be optionally substituted by from one to three more groups selected from halogen, hydroxyl, amino, nitro, trifluoromethyl, trifluoromethoxy, alkyl, alkenyl, alkynyl, cyano, carboxy, carboalkoxy, aralkyl, aralkenyl or aralkynyl, 1,2-dioxymethylene, 1,2-dioxyethylene, alkoxy, alkenoxy, alkynoxy, aralkoxy, aryl-substituted alkenoxy or aryl substituted alkynoxy, alkylamino, alkenylamino or alkynylamino, aryl substituted alkylamino, aryl substituted alkenylamino, aryl substituted alkynylamnino, aryl substituted carbonyloxy, alkylcarbonyloxy, aliphatic or aromatic acyl, aryl substituted acyl, arylsubstituted alkylcarbonyloxy, aryl substituted carbonylamino, aryl substituted amino, aryloxy, arylcarbonyl, alkylcarbonylarnino, arylsubsubstituted alkylcarbonylamino, alkoxycarbonylamino, aryl substituted alkoxycarbonylamino, aryl oxycarbonylamino, alkylsuphonylamino, mono- or bis-(aryl sulphonyl) amino, aralkylsulphonylamino, morpholinocarbonylamino, thiomorpholinocarbonylamino, N-alkyl guanidino, N,N-dialkylguanidino, N,N,N-trialkylguanidino, N-alkyl urea, N,N-dialkylurea, N-arylurea, N,N,(alkyl aryl) urea, N,N-(diaryl) urea, aralloxycarbonyl-substituted alkyl, aralkylamino carbonyl, or thioaryloxy and where any aryl group listed above, may itself be further substituted by up to three substitutents selected from halogen, hydroxyl, amino, nitro, trifluoromethyl, trifluoromethoxy, alkyl, alkenyl alynyl, 1,2-dioxymethylene, 1,2-dioxyethylene, alkoxy, alkenoxy, alkynoxy, alkylamino, alkenylamino or alkynylamino, alkylcarbonyloxy, aliphatic or aromatic acyl, alkylcarbonylamino, arylsubsubstituted alkylcarbonylamino, alkoxycarbonylamino, alkylsuphonylamino, N-alkyl urea or N,N-dialkylurea.

A particular example of D is a group of sub-formula (iii)

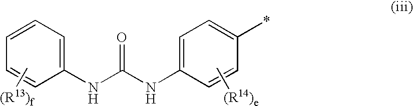

where * is the point of attachment to the group X in formula (I), each R¹³ or R¹⁴ is independently selected from C₁₋₆alkyl, C₂₋₆alkenyl, C₂₋₆alkynyl, C₁₋₄alkoxy, C₁₋₄ alkanoyl, C₁₋₆alkylamino, C₁₋₄alkoxylC₁₋₆alkyl, C₁₋₆alkylaminoC₁₋₆alkyl, cyano, nitro, halogeno, trifluoromethyl, hydroxy, (CH₂)ₚOH where p is 1 or 2, —CO₂R^{e5}, and —CONR^{e5}R^{f5}, where R^{e5} and R^{f5} are independently hydrogen and C₁₋₆alkyl, or two adjacent substituents can be taken together to form a 5-7 membered ring; and

f and e are independently selected from zero or an integer from 1 to 5.

Suitably f and e are independently selected from zero o f an integer of from 1 to 3, and most preferably, they are 1.

The 5 to 7 membered ring formed by adjacent substituents R¹³ can be an, optionally substituted, saturated or unsaturated ring with up to three heteroatoms independently selected from nitrogen, oxygen and sulphur. Suitable optional substitutents include those listed above for heterocycles

In a particularly preferred embodiment of the invention, there is provided a compound of formula (II)

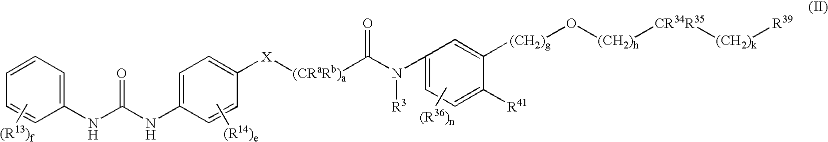

wherein X R^{a}, R^{b}, a, R³, R³⁶, n, R⁴¹, g, h, R³⁴, R³⁵, k, R³⁹ and R⁴¹ are as defined above in relation to formula (I) and R¹³, R¹⁴ e and f are as defined above in relation to sub-formula (iii).

Preferably f is 1 and R¹³ is C₁₋₆alkyl, such as methyl. Preferably e is 0 or 1 and R¹⁴ is C₁₋₄alkoxy such as methoxy. In particular the compound of formula (II) is a compound of formula (III)

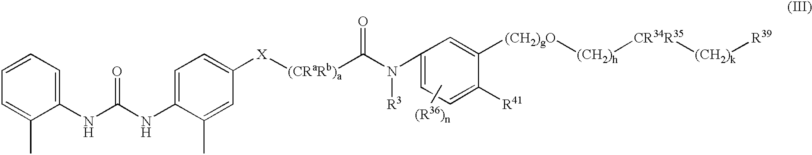

wherein, X, R^{a}, R^{b}, a, R³, R³⁶, n, R⁴¹, g, h, R³⁴, R³⁵, k, R³⁹ and R⁴¹ are as defined above; and R¹⁵ is hydrogen or C₁₋₄alkoxy.

Preferably, X is a direct bond or oxygen, and most preferably a direct bond.

Suitably R^{a} and R^{b} are independently selected from hydrogen or C₁₋₂ alkyl and preferably they are both hydrogen.

Suitably a is 1 or 2 and preferably 1.

Preferably R³ is hydrogen or C₁₋₂ alkyl, more preferably hydrogen.

Preferably R³⁹ is carboxy.

Preferably n is 0 or 1 and most preferably n is 0. Where a group R³⁶ is present, it is suitably C₁₋₆ alkyl or C₁₋₄ alkoxy, such a methoxy.

In a preferred embodiment R²⁴ and R³⁵ are independently selected from hydrogen or C₁₋₆alkyl such as methyl.

Preferably h and k are 1.

Preferably g is 0.

As mentioned above, R⁴¹ is a group of formula V

U - (CH₂)_{d} - V - T (V)

as defined above.

A particularly preferred group U is oxygen.

Most preferably V is a direct bond or is sulphur, SO₂ or oxygen.

d is suitably 0 or 2 or 3.

Suitably, T is a heterocycle, in particular a nitrogen containing heterocycle which may be optionally substituted as defined above.

Particularly preferred examples of the group R⁴¹ include

(i) the group C₁₋₄alkoxyC₁₋₄akoxyl, such as 2-methoxyethoxy.

ii) the group O—(CH₂)_{d}—T where d is 0 or an integer of 1 to 4, preferably 2 or 3, and T is a nitrogen containing heterocycle linked to oxygen through a ring nitrogen or ring carbon and preferably is selected from piperidinyl, morpholinyl, piperaznyl, methylpiperazinyl, pyrrolidinyl, imidazolyl and pyridyl;

iii) the group —O—(CH₂)_{d}—N—R^{c}R^{d}, where d is 2 or 3, R^{c} and R^{d} are each independently (CH₂)₂OCH₃, C₁₋₄ alkyl or hydrogen;

iv) R⁴¹ is the group —O—(CH₂)_{d}—T where d is from 1 to 4, preferably 2 or 3 and T is a nitrogen containing heterocycle linked to oxygen through a ring nitrogen and preferably is selected from piperidinyl, morpholinyl, imidazolyl, pyrrolidinyl and pyridyl; or

v) R⁴¹ is the group —(CH₂)_{d}—T where d is 1 and T is morpholinyl.

Particularly suitable compounds are those described in the Examples and in Table 1.

TABLE 1

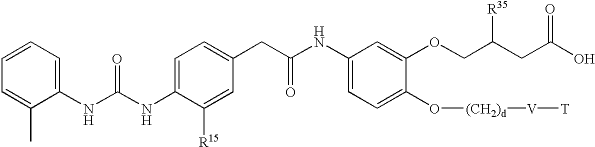

| Compd No. | R³⁵ | d | V | R¹⁵ | T |
|---|---|---|---|---|---|
| 1 | H | 2 | bond | H | 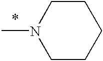 |
| 2 | H | 3 | bond | H | 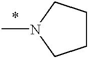 |
| 3 | H | 3 | bond | H | 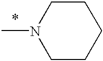 |
| 4 | CH₃ | 3 | bond | H | 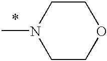 |
| 5 | CH₃ | 3 | bond | H | 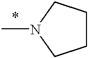 |
| 6 | CH₃ | 3 | bond | H | 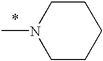 |
| 7 | CH₃ | 0 | bond | H | 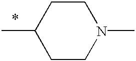 |
| 8 | H | 0 | bond | H | 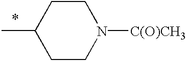 |
| 9 | H | 0 | bond | H | 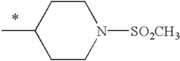 |
| 10 | CH₃ | 0 | bond | H | 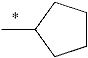 |
| 11 | CH₃ | 0 | bond | H | 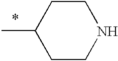 |
| 12 | H | 3 | O | OCH₃ | benzyl |
| 13 | H | 3 | O | OCH₃ | H |
| 14 | H | 3 | S | OCH₃ | CH₃ |
| 15 | CH₃ | 0 | bond | H | 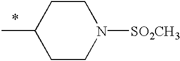 |
| 16 | CH₃ | 0 | bond | H | 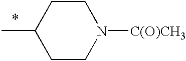 |
| 17 | H | 3 | SO₂ | —OCH₃ | CH₃ |

In the above Table, * indicates the point of attachment to the —O(CH₂)_{d}V— group.

Pharmaceutically acceptable salts include acid addition salts such as salts formed with mineral acids, for example, hydrogen halides such as hydrogen chloride and hydrogen bromide, sulphonic and phosphonic acids; and salts formed with organic acids, especially citric, maleic, acetic, oxalic, tartaric, mandelic, p-toluenesulphonic, methanesulphonic acids and the like. In another aspect, suitable salts are base salts such as alkali metals salts, for example, sodium and potassium; alkae earth metal salts such as magnesium and calcium; aluminium and ammonium salts; and salts with organic bases such as ethanolamine, methylamine, diethylamine, isopropylamine, trimethylamine and the like. Such salts may be prepared by any suitable method known in the art.

In vivo hydrolysable derivatives include, in particular, pharmaceutically acceptable derivatives that may be oxidised or reduced in the human body to produce the parent compound or esters that hydrolyse in hte human body to produce the parent compound. Such esters can be identified by administering, for example, intravenously to the test animal, the compound under test and subsequently examining the test animal's body fluids. Suitable in vivo hydrolysable esters for hydroxy include acetyl and for carboxyl include, for example, alkyl esters, dialkylaminoalkoxy esters, esters of formula —C(O)—O—CH₂C(O)NR^{a″}R^{b″} where R^{a″} and R^{b″} are, for example, selected from hydrogen and C₁₋₄ alkyl, and C₁₋₆alkoxy methyl esters for example methoxymethyl, C₁₋₆alkanoyloxymethyl esters for example pivaloyloxymethyl, phthalidyl esters, C₃₋₈ cycloalkoxycarbonyloxyC₁₋₆alkyl esters for example 1-cyclohexylcarbonyloxyethyl; 1,3-dioxolan-2-ylmethyl esters for example 5-methyl-1,3-dioxolan-2-ylmethyl; and C₁₋₆alkoxycarbonyloxyethyl esters for example 1-methoxycarbonyloxyethyl.

The activities of the compounds of this invention to inhibit the interaction between VCAM-1 and fibronectin with integrin α₄β₁ may be determined using a number of in vitro and in vivo screens.

For example, compounds of formulae (I), (II), or (III) preferably have an IC₅₀ of <10 μM, more preferably <1 μM in the MOLT-4 cell/Fibronectin assay hereinafter described.

In order for it to be used, a compound of formulae (I), (II) or (III) or a pharmaceutically acceptable salt or an in vivo hydrolysable derivative thereof is typically formulated as a pharmaceutical composition in accordance with standard pharmaceutical practice.

Thus, according to a further aspect of the invention there is provided a pharmaceutical composition which comprises a compound of formulae (I), (II) or (III) or a pharmaceutically acceptable salt or an in vivo hydrolysable derivative thereof and a pharmaceutically acceptable carrier.

The pharmaceutical compositions of this invention may be in a form suitable for oral use, for example a tablet, capsule, aqueous or oily solution, suspension or emulsion; for nasal use, for example a snuff, nasal spray or nasal drops; for vaginal or rectal use, for example a suppository; for administration by inhalation, for example as a finely divided powder or a liquid aerosol; for sub-lingual or buccal use, for example a tablet or capsule; or for parenteral use (including intravenous, subcutaneous, intramuscular, intravascular or infusion), for example a sterile aqueous or oily solution or suspension, or a depot formulation with drug incorporated in a biodegradable polymer. The composition may be in a form suitable for topical administration such as for example creams, ointments and gels. Skin patches are also contemplated. For these purposes, the compositions of this invention may be formulated by means known in the art, such as for example, as described in general terms, in Chapter 25.2 of Comprehensive Medicinal Chemistry, Volume 5, Editor Hansch et al, Pergamon Press 1990.

Furthermore, the pharmaceutical composition of the present invention may contain one or more additional pharmacological agents suitable for treating one or more disease conditions referred to hereinabove, in addition to the compounds of the present invention. In a further aspect, the additional pharmacological agent or agents may be co-administered, either simultaneously or sequentially, with the pharmaceutical compositions of the invention.

The composition of the invention will normally be administered to humans such that the daily dose will be 0.01 to 75 mg/kg body weight and preferably 0.1 to 15 mg/kg body weight. A preferred composition of the invention is one suitable for oral administration in unit dosage form for example a tablet or capsule which contains from 1 to 1000 mg and preferaly 10 to 500 mg of a compound according to the present invention in each unit dose.

Thus, according to yet another aspect of the invention, there is provided a compound of formulae (I), (II), or (III) or a pharmaceutically acceptable salt or an in vivo hydrolysable derivative thereof for use in a method of therapeutic treatment of the human or animal body.

In yet a further aspect of the invention the present invention provides a method of treating a disease mediated by the interaction between VCAM-1 and/or fibronectin and the integrin receptor α₄β₁ in need of such treatment which comprises administering to said warm-blooded mammals an effective amount of a compound of formulae (I), (II), or (III) or a pharmaceutically acceptable salt or an in vivo hydrolysable derivative thereof.

The present invention also provides the use of a compound of formulae (I), (II), or (III) or a pharmaceutically acceptable salt or an in vivo hydrolysable derivative thereof in the production of a medicament for use in the treatment of a disease or medical condition mediated by the interaction between fibronectin and/or VCAM-1 (especially VCAM-1) and the integrin receptor α₄β₁.

In a preferred embodiment the mammal in need of treatment is suffering from multiple sclerosis, rheumatoid arthritis, asthma, coronary artery disease, psoriasis, atherosclerosis, transplant rejection, inflammatory bowel disease, insulin-dependent diabetes and glomerilonephritis.

Compounds of formula (I) may be prepared by conventional routes, for example as described in WO 98/04247. In particular, they may be prepared by process which comprises coupling together a compound of formula (VI)

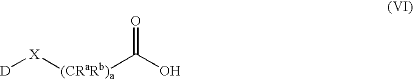

where D, X, R^{a}, R^{b} and a are as defined hereinbefore in relation to formula (I); and an appropriate amine of formula (VII)

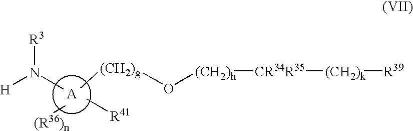

where R³, A, R³⁶, n, R⁴¹, g, h, R³⁴ R³⁵, k and R³⁹ are as hereinbefore defined in relation to formula (I); and

where any functional group is optionally protected;

and thereafter, if necessary:

a) removing any protecting group; and

b) forming a pharmaceutically acceptable salt or in vivo hydrolysable derivative.

The reactions to couple the acids of formula (VI) to the amines of formula (VII) are to are suitably performed under standard coupling conditions for forming peptide bonds. They can be performed either on a solid support (Solid Phase Peptide Synthesis) or in solution using normal techniques used in the synthesis of organic compounds. With the exception of the solid support, all the other protecting groups, coupling agents, deblocking reagents and purification techniques are similar in both the solid phase and solution phase peptide synthesis techniques.

During the reaction, amino acid functional groups may, if necessary, be protected by protecting groups, for example BOC (tert-butoxycarbonyl). Such groups can be cleaved when necessary using standard techniques such as acid or base treatment.

Suitable protecting groups for the protection of the carboxyl groups include esters.

Coupling reagents for forming peptide bonds include the commonly used azide, symmetrical anhydride, mixed anhydride and various active esters and carbodiimides. In the case of carbodiimides, additives such as 1-hydroxybenzotriazole and N-hydroxysuccninide may also be added. Other coupling reagents include 1H-benzotriazole-1-yloxy-tris-pyrrolidinophosphonium hexafluorophosphate (PyBOP), (2-(1H-benidtriazole-1-yl)1,1,3,3-tetramethyluronium tetrafuoroborate (TBTU), (2-(1H-benzotriazole-1-yl)1,1,3,3-tetramethyluronium hexafluorophosphate (HBTU)] and O-(7-azabenzotriazol-1-yl)1,1,3,3-tetramethyluronium hexafluorophosphate (HATU).

The coupling reactions can be performed at temperatures between −20° C. to 40° C. The time of the reaction can vary such as between 10 minutes and 24 hours.

Suitable purification methods for the intermediates and final products include chromatographic techniques such as high pressure liquid chromatography (HPLC) along with many other standard techniques used in organic chemistry (e.g. solvent extraction and crystallisation).

It will be understood that all amino acids are the natural isomers unless otherwise stated. The following examples illustrate the invention.

### EXAMPLE 1

### Method for Alkylation of 4-nitrocatechol to Give Nitrophenol (18)

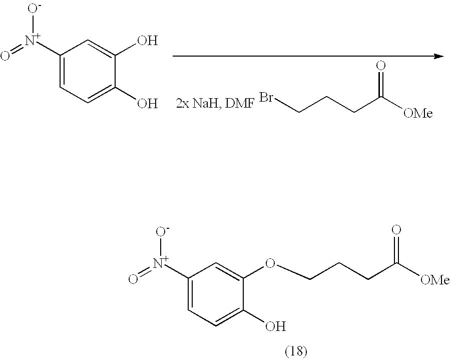

Sodium hydride (5.2 g of a 60% suspension in oil; 129 mmol) was suspended in DMF (50 cm³) and cooled to 0° C. 4-Nitrocatechol (10 g; 64.5 mmol) in DMF (50 cm³) was slowly added. The purple solution that formed was stirred for 0.5 h and then methyl 4-bromobutyrate (7.4 cm³; 64.5 mmol) was added. The reaction mixture was very viscous for about 1 h but after this time it became quite fluid. The mixture was stirred for 24 h by which time it had become red in colour. The reaction mixture was quenched by careful addition of water and then acidified with 2N hydrochloric acid causing the mixture to tumn pale yellow. The mixture was extracted with ethyl acetate (×2) and the combined extracts,washed with brine (×2), saturated, aqueous sodium bicarbonate, brine, dried (MgSO₄) and concentrated under reduced pressure. The yellow sticky solid was washed with hexane to remove mineral oil and gave the nitrophenol (18) (15 g, 67%) as a pale yellow solid.

MS (ES+) 256.4 (M+H)⁺

¹H (300 MHz; CDCl₃) 2.20 (2H, m), 2.55 (2H, t), 3.75 (3H, s), 4.20 (2H, t), 6.60 (1H, brs), 6.95 (1H, d), 7.75 (1H, d), 7.90 (1H, dd).

### Alkylation of Nitrophenol (18)

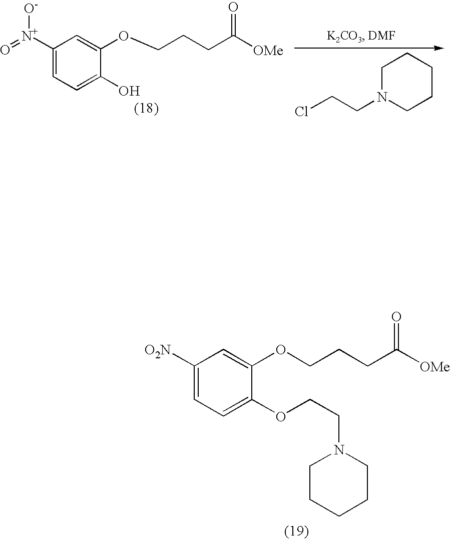

Nitrophenol (18) (0.75 g; 2.9 mmol), N-(2-chloroethyl)-piperidine hydrochloride (0.63 g; 3.4 mmol) and potassium carbonate (1.1 g; 7.9 mmol) were heated to 80° C. in DMF over night. After cooling, the mixture was concentrated under reduced pressure. The residue was suspended between DCM and 1N sodium hydroxide, and the organic phase was separated, washed with brine and passed through phase separating paper. The solvent was removed under reduced pressure to give crude nitrobenzene (19) which was used without further purification MS ES+ 367.1 (M+H)

### Reduction of Nitrophenol (19)

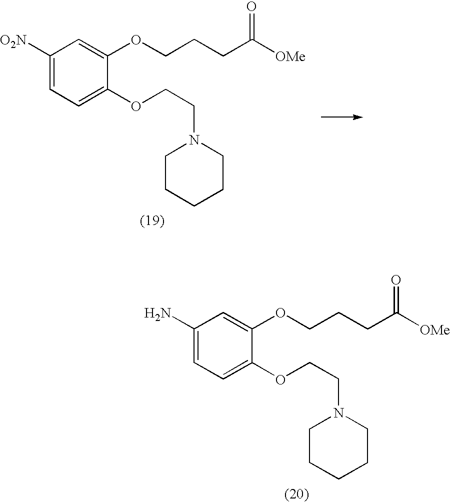

To the entire sample of nitrophenol (19) was added methanol and ammonium formate (1 g), and the system was purged with argon. To this was added 10% Pd on charcoal (25 mg) and the mixture was heated to 45° C. for 1 hour. The mixture was filtered through a pad of celite and the solvent was removed under reduced pressure. The residue was suspended between DCM and aq.satNaHCO₃. The organic phase was separated, washed with brine and passed through phase separating paper. The solvent was removed under reduced pressure to give crude anline (20) which was used without further purification. MS ES+ 337.1 M+H)

### Preparation of Ester (22)

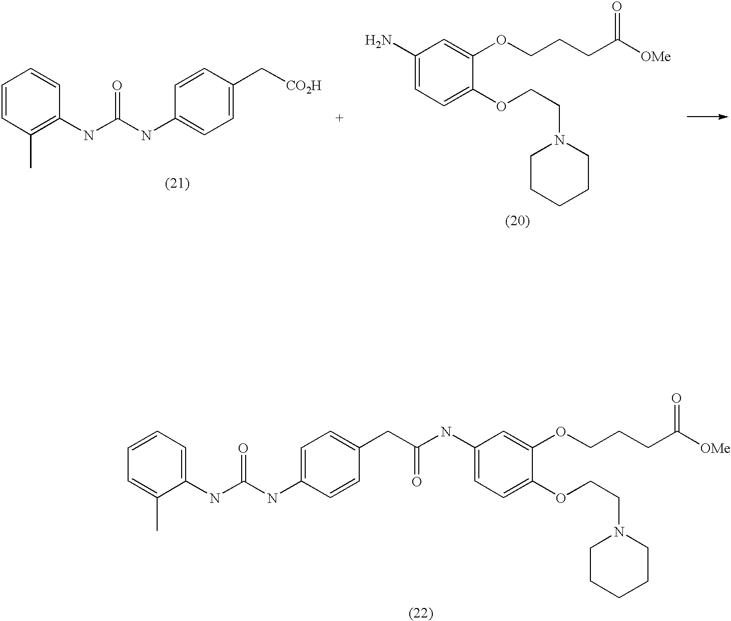

To a DMF solution (3 ml) of crude aniline (20) (300 mg) was added diphenylurea phenylacetic acid (21) (prepared according to the method described in WO97/03094) and HATU (O-(7-azabenzotriazole-1-yl)-1,1,3,3-tetramethyluronium hexafluorophosphate, 310 mg, 0.82 mmol). The system was purged with argon then DIPEA (diisopropylethylamine, 0.33 ml) was added. The mixture was stirred for 72 hours, and then quenched with water and DCM. The organic phase was washed with aq.satNaHCO₃ and brine, then passed through phase separating paper. The solvent was removed under reduced pressure to give ester (22) as a crude solid which was used without further purification. MS ES+ 603 (M+H).

### Hydrolysis of Ester (22) to give Compound 1 in Table 1

To a solution in THF (12 ml) of the entire sample of ester (22) was added water (5 ml) and LiOH.H₂O (205 mg, 5 mmol) and the mixture was allowed to stir for 12 hours. The mixture was acidified with aq. 2NHCl (5 ml) and the solvent was removed under reduced pressure to give a sticky solid. The solid was taken up in warm MeOH and purified using an Isolute SCX column (eluant 1% ammonia in MeOH) and the solvent was removed. The solid was dried in a vacuum oven to give Compound 1 as a solid (140 mg).

¹H NMR (DMSO; 300 MHz) 1.35 (2H, d), 1.45 (4H, m), 1.90 (2H, t), 2.23 (3H, s), 2.45 (4H, m), 2.60 (2H, t), 3.50 (2H, s), 3.95 (2H, t), 4.00 (2H, t), 6.90 (2H, m), 7.05-7.25 (6H, m), 7.45 (2H, d), 7.75 (1H, d), 8.60(1H, s), 9.70 (1H, s), 9.95 (1H, s).

MS ES+ 589.5 (M+H), ES− 587.4 (M−H).

### EXAMPLE 2

### Preparation of Nitro Compound (23)

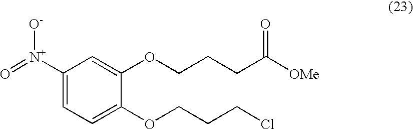

Nitrophenol (18) (2.5 g; 9.80 mmol), 1-bromo-3-chloropropane (1.9 cm³; 19.6 mmol) and caesium carbonate (9.6 g; 29.4 mmol) were heated under reflux in acetone (120 cm³) over night. After cooling, the mixture was filtered and concentrated under reduced pressure. The residue was dissolved in DCM, washed with 2N sodium hydroxide, brine, dried (MgSO₄) and concentrated under reduced pressure to give a crystalline solid. This was suspended in iso-hexane and filtered to give the product (23) as a colourless crystalline solid (3.1 g, 95%).

¹H NMR (CDCl₃; 300 MHz) 2.20 (2H, m), 2.30 (2H, m), 2.55 (2H, t), 3.70 (3H, s), 3.80 (2H, t), 4.15 (2H, t), 4.25 (2H, t), 6.95 (1H, d), 7.75 (1H, d), 7.90 (1H, dd).

### Preparation of Aniline (24)

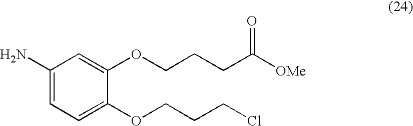

Nitro compound (23) (1.5 g; 4.52 mmol), iron powder (1.5 g; 27.1 mmol) and ammonium chloride (0.17 g; 3.16 mmol) were heated under reflux in ethanol/water (30/9.9 cm³) for 1 h After cooling, the mixture was diluted with ethyl acetate and filtered. The filtrate was dried (MgSO₄) and concentrated under reduced pressure to give the aniline (24) as a pale brown oil (1.06 g, 78%).

¹H NMR (CDCl₃; 300 MHz) 2.15 (4H, m), 2.55 (2H, t), 3.70 (3H, s), 3.75 (2H, t), 4.00 (2H, t), 4.05 (2H, t), 6.20 (1H, dd), 6.30 (1H, d), 6.75 (1H, d).

### Preparation of Amide (25)

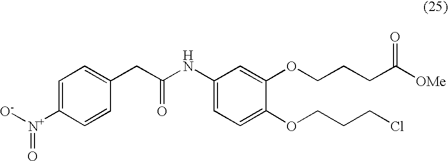

Aniline (24) (960 mg; 3.18 mmol), 4-nitrophenylacetic acid (692 mg; 3.82 mmol), HATU (O-(7-azabenzotriazole-1-yl)-1,1,3,3-tetramethyluronium hexafluorophosphate, 1.4 g; 3.82 mmol) were dissolved in DMF (5 cm³) and DIPEA (diisopropylethylamine. 1.4 cm³; 7.95 mmol) added. The reaction mixture was stirred at room temperature for 16 h. The mixture was diluted with ethyl acetate, washed with 2N aqueous hydrochloric acid, 2N sodium hydroxide, brine, dried (MgSO₄) and concentrated under reduced pressure. The residue was chromatographed (1% MeOH/DCM) to give the product amide (25) as a pale brown solid (852 mg, 58%).

¹H NMR (CDCl₃; 300 MHz) 2.10 (2H, m), 2.20 (2H, m), 2.55 (2H, t), 3.70 (3H, s), 3.80 (4H, m), 4.00 (2H, t), 4.10 (2H, t), 6.85 (2H, s), 7.05 (1H, s), 7.55 (2H, d), 8.25 (2H, d).

### Preparation of Ester (26)

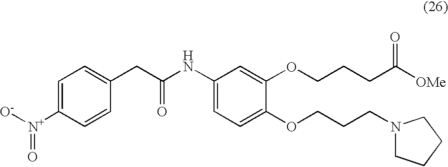

Amide (25) (275 mg; 0.59 mmol) and pyrrolidine (99 ml; 1.18 mmol) were heated at 120° C. in O-dichlorobenzene (3 cm³) for 5 h. The mixture was cooled and placed on top of a silica column. The product was then eluted with MeOH/DCM (5-7%) to give the product ester (26) as a brown oil (260 mg, 88%).

¹H NMR (CDCl₃; 300 MHz) 1.95 (4H, m), 2.10 (2H, m), 2.25 (2H, m), 2.50 (2H, t), 3.00 (6H, m), 3.65 (3H, s), 3.90 (4H, m), 3.95 (2H, t), 6.70 (1H, d), 6.90 (1H, dd), 7.50 (1H, d), 7.60 (2H, d), 8.15 (2H, d), 8.75 (1H, brs).

### Preparation of Aniline (27)

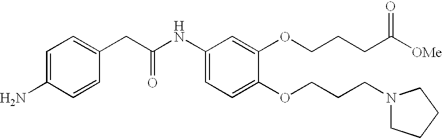

Ester (26) (260 mg; 0.52 mmol) was dissolved in MeOH (3 cm³) and the system purged with argon gas. 10% Pd/C (26 mg; 10% by mass ) was added and the mixture stirred under a hydrogen atmosphere for 3 h. The Pd/C was removed by filtration and the filtrate concentrated under reduced pressure to give the product aniline (27) as a pale brown oil (250 mg, 100%) MS 470.4 M+H)⁺

### Preparation of Ester (28)

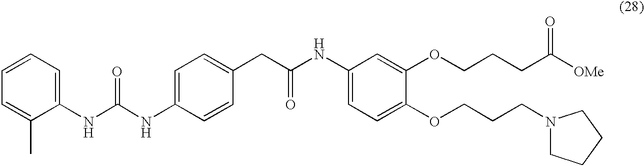

Aniline (27) (250 mg; 0.53 mmol) was dissolved in DMF (5 cm³) and o-tolyl isocyanate (79 ml; 0.64 mmol) added. The mixture was stirred at room temperature for 16 h. The mixture was diluted with ethyl acetate and washed with brine. This caused a sticky gum to form on the inside of the separating funnel which was dissolved in MeOH and placed on an Isolute SCX column. After washing with MeOH the product was eluted with 1% NH₃/MeOH to give a pale brown solid (130 mg, 41%).

¹H NMR (DMSO; 300 MHz) 1.65 (4H, m), 1.85 (2H, m), 1.95 (2H, m), 2.25 (3H, s), 2.40 (4H, m), 3.50 (2H, s), 3.60 (3H, s), 3.90 (4H, m), 6.90 (2H, m), 7.05-7.15 (3H, m), 7.20 (2H, d), 7.30 (1H, d), 7.40 (2H, d),,7.80 (1H, d), 7.85 (1H, s), 8.95 (1H, s), 9.90 (1H, s).

### Hydrolysis of Ester (28) to give Compound 2 in Table 1

This was carried out following the procedure described above for hydrolysis of ester (22) to gve Compound 2 as a pale yellow foam.

MS 589.5 (M+H)⁺

¹H NMR (DMSO; 300 MHz) 1.55 (4H, m), 1.85 (4H, m), 2.10 (2H, t), 2.25 (3H, s), 2.40 (4H, m), 3.50 (2H, s), 3.90 (4H, m), 6.85 (1H, d), 6.90 (1H, t), 7.10 (5H, m), 7.25 (1H, d), 7.45 (2H, d), 6.90 (1H, t), 7.10 (5H, m), 7.25 (1H, d), 7.45 (2H, d), 7.70 (1H, d), 9.10 (1H, s), 9.90 (1H, s), 10.20 (1H, brs)

HPLC 11.75 min

### EXAMPLE 3

### Preparation of Ester (29)

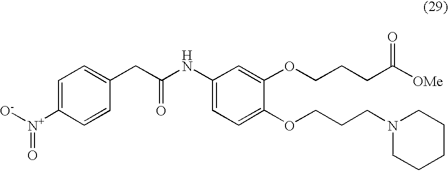

Using the procedure described for formation of ester (26), amide (25) was treated with piperidine to give ester (29).

Ester (29) was then treated in an analogous fashion to ester (26) to give Compound 3 in Table 1.

MS 603.4 (M+H)⁺

¹H NMR (DMSO; 300 MHz) 1.35 (2H, m), 1.50 (4H, m), 1.80 (2H, m), 1.90 (2H, m), 2.25 (3H, s), 2.35 (8H, m), 3.50 (2H, s), 3.90 (4H, m), 6.85 (1H, d), 6.90 (1H, t), 7.10 (3H, m), 7.20 (2H, d), 7.30 (1H, d), 7.40 (2H, d), 7.80 (1H, d), 8.10 (1H, s), 9.95 (1H, s) HPLC 12.10 min

### EXAMPLE 4

### Synthesis of (±) Methyl 4-bromo-3-methylbutyrate

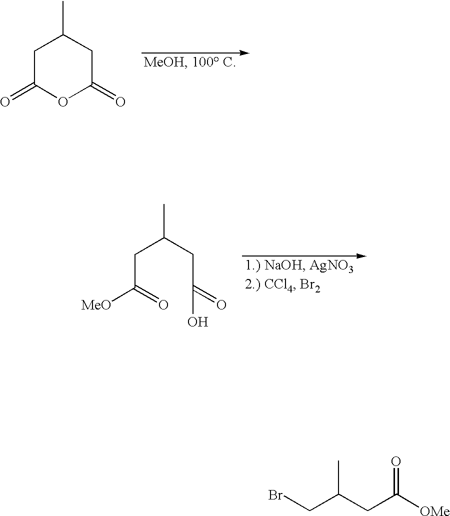

3-Methylglutaric anhydride (50 g; 390 mmol) and dry methanol (15.8 cm³; 390 mmol) were heated to reflux (≈100° C.). After 1 h the mixture stopped refluxing but was maintain at 100° C. over night. After cooling, the mixture was distilled (≈95° C. at 0.2 mmHg) to give monomethyl 3-methylglutaric acid (37 g, 59%) as a colourless oil.

MS (ES−) 159.0 (M−H)⁻. (ES+) 161.0 (M+H)⁺ [175.1 (M+H)⁺ for diester].

¹H NMR (300 MHz; CDCl₃) 1.10 (3H, d), 2.30 (2H, m), 2.45 (3H, m), 3.70 (3H, s).

Monomethyl 3-methylglutaric acid (37 g; 231 mmol) was added to 1M NaOH (231 cm³; 231 mmol) causing the solution to warm slightly. This solution was added to a solution of silver nitrate (39.2 g; 231 mmol) in water (184 cm³) at ≈60° C. A fine white precipitate form immediately. The mixture was cooled and stirred in an ice bath for 1 h before being filtered, washed with water, acetone and ether and partially dried on the filter. The solid was then dried over night at 80° C. in a vacuum oven to give the silver salt of the monomethyl 3-glutaric acid (49 g, 79%) as a pale brown solid.

The silver salt (49 g; 184 mmol) was suspended in carbon tetrachloride (245 cm³) and bromine (9.5 cm³) slowly added. The reaction mixture warmed to ≈30° C. during this process an effervescence was seen. The reaction mixture was maintained at this temperature by the rate of addition of bromine. After the final addition of bromine the viscous mixture was stirred for 0.5 h before being heated at reflux for 1 h. After cooling, the pale yellow precipitate was removed by filtration and the filtrate washed with 1M aqueous sodium thiosulphate, brine, dried (phase separation paper) and concentrated under reduced pressure. This gave a pale yellow oil which contained 15% of moniomethyl 3-methyl glutaric acid as an impurityby ¹H NMR. This was removed by taking the oil up in DCM and washing with 1M NaOH. Drying and concentration as above gave (±) methyl 4-bromo-3-methylbutyrate (25 g, 70%) as a pale yellow oil.

¹H NMR (300 MHz; CDCl₃) 1.10 (3H, d), 2.30 (2H, m), 2.55 (1H, dd), 3.40 (1H, dd), 3.45 (1H, dd), 3.70 (3H, s).

### Preparation of Nitrophenol (30)

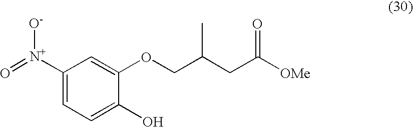

Using the procedure described for formation of nitrophenol (18), nitrocatechol was alkylated with (±) methyl 4bromo-3-methylbutyrate to give nitrophenol (30).

### Preparation of Nitro Compound (31)

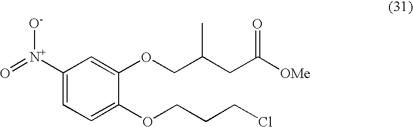

Using the procedure described for formation of nitro compound (23), nitrophenol (30) was alkylated with 1-bromo-3-chloropropane to give nitro compound (31).

### Preparation of Nitro Compound (32)

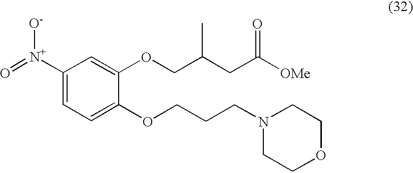

Using the procedure described for formation of ester (26), nitro compound (31) was treated with morpholine to give nitro compound (32).

### Preparation of Aniline (33)

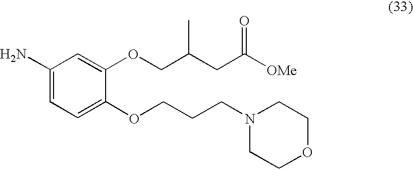

Using the procedure described for preparation of aniline (27), nitro compound (32) was reduced with hydrogen and Pd to give aniline (33).

### Preparation of Ester (34)

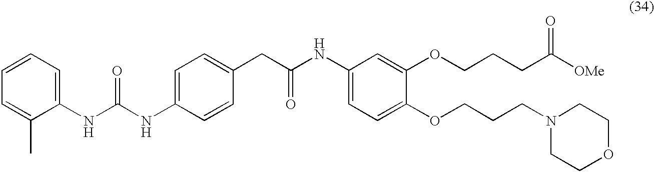

Using the procedure described for preparation of ester (22), aniline (33) was coupled with diphenylurea phenylacetic acid (21) to give ester (34).

### Preparation of Compound 4 in Table 1

Using the procedure described for the hydrolysis of ester (22), ester (34) was hydrolysed to give Compound 4.

MS 619.5 (M+H)⁺

¹H NMR (DMSO; 300 MHz) 1.00 (3H, d), 1.80 (2H, m), 1.95 (1H, dd), 2.15-2.35 (9H, m), 2.40 (2H, t), 3.50 (2H, s), 3.55 (4H, t), 3.70 (1H, dd), 3.85 (1H, dd), 3.95 (2H, t), 6.85 (1H, d), 6.90 (1H, t), 7.05-7.20 (5H, m), 7.25 (1H, d), 7.45 (2H, d), 7.70 (1H, d), 8.95 (1H, s), 9.90 (1H, s), 10.05 (1H, brs)

HPLC 11.94 min

### EXAMPLE 5

### Preparation of Nitro Compound (35)

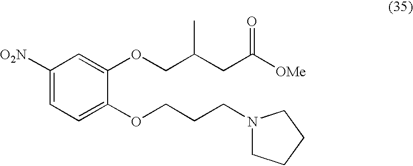

Using the procedure described for formation of ester (26), nitro compound (31) was treated with pyrrolidine to give nitro compound (35).

### Preparation of Aniline (36)

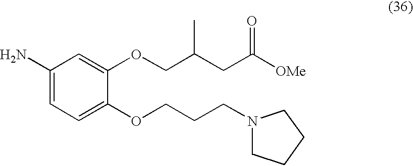

Using the procedure described for preparation of aniline (27), nitro compound (35) was reduced with hydrogen and Pd to give aniine (36).

### Preparation of Ester (37)

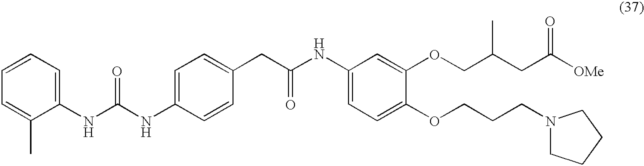

Using the procedure described for preparation of ester (22), aniline (36) was coupled with diphenylurea phenylacetic acid (21) to give ester (37).

### Preparation of Compound 5 in Table 1

Using the procedure described for the hydrolysis of ester (22), ester (37) was hydrolysed to give Compound 5.

MS 603.5 (M+H)⁺

¹H NMR (DMSO; 300 MHz) 1.00 (3H, d), 1.65 (4H, m), 1.80 (2H, m), 2.00 (1H, m), 2.25 (5H, m), 2.40 (4H, m), 2.55 (2H, t), 3.50 (2H, s), 3.70 (1H, dd), 3.85 (1H, dd), 3.90 (2H, t), 6.85 (1H, d), 6.90 (1H, t), 7.05-7.20 (5H, m), 7.25 (1H, d), 7.45 (2H, d), 7.70 (1H, d), 8.80 (1H, s), 9.90 (2H, brs)

HPLC 12.33 min

### EXAMPLE 6

### Peparation of Nitro Compound (38)

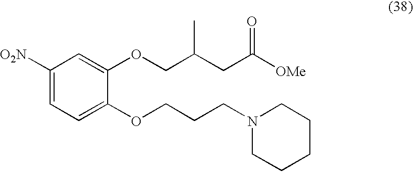

Using the procedure described for formation of ester (26), nitro compound (31) was treated with piperidine to give nitro compound (38).

### Preparation of Aniline (39)

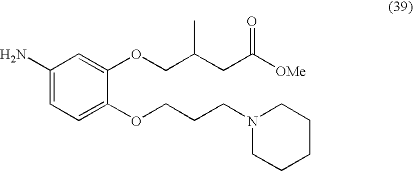

Using the procedure described for preparation of aniline (27), nitro compound (38) was reduced with hydrogen and Pd to give aniline (39).

### Preparation of Ester (40)

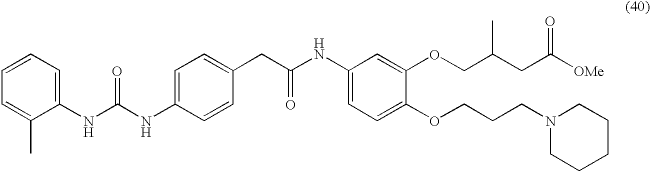

Using the procedure described for preparation of ester (22), aniline (39) was coupled with diphenylurea phenylacetic acid (21) to give ester (40).

### Preparation of Compound 6 in Table 1

Using the procedure described for the hydrolysis of ester (22), ester (40) was hydrolysed to give Compound 6.

MS 617.6 (M+H)⁺

¹H NMR (DMSO; 300 MHz) 1.00 (3H, d), 1.35 (2H, m), 1.45 (4H, m), 1.80 (2H, m), 1.95 (1H, dd), 2.15-2.40 (11H, m), 3.50 (2H, s), 3.65 (1H, dd), 3.90 (3H, m), 6.85 (1H, d), 6.90 (1H, t), 7.05-7.20 (5H, m), 7.25 (1H, d), 7.45 (2H, d), 7.70 (1H, d), 9.00 (1H, s), 9.90 (1H, s), 10.05 (1H, brs)

HPLC 12.70 min

### EXAMPLE 7

### Preparation of Nitro Compound (41)

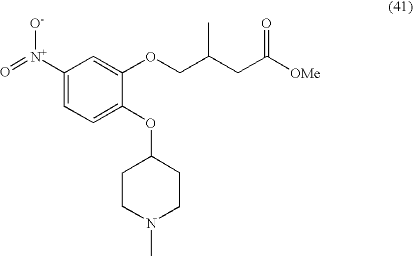

Nitro phenol (30) (1 g; 4.0 mmol), N-Methyl-4-hydroxypiperidine (0.46 g, 4 mmol) and triphenylphosphine (1.13 g; 4.31 mmol) were dissolved in THF (15 cm³) and cooled to 0° C. Diethyl azodicarboxylate (0.7 cm³; 4.31 mmol) was added dropwise and after the final addition the mixture allowed to warm to room temperature and stirred over night. The solvent was removed under reduced pressure and the residue taken-up in DCM, washed with 2N aqueous hydrochloric acid, 2N sodium hydroxide, dried (MgSO₄) and concentrated under reduced pressure. The residue was chromatographed (DCM - 1% MeOH/DCM) to give the product nitro compound (41) as a viscous yellow oil.

### Preparation of Aniline (42)

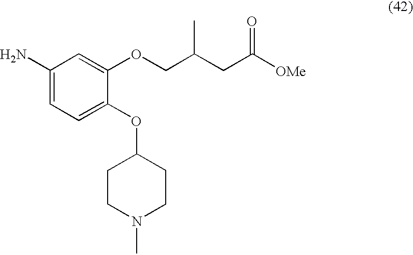

Using the procedure described for preparation of aniline (27), nitro compound (41) was reduced with hydrogen and Pd to give aniline (42).

### Preparation of Ester (43)

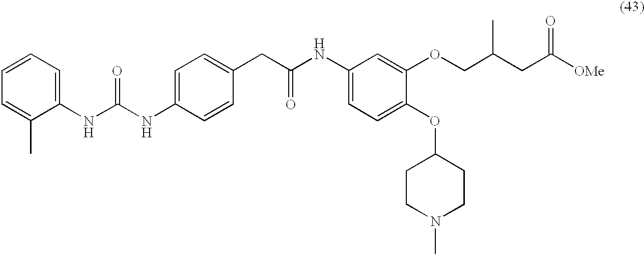

Using the procedure described for preparation of ester (22), aniline (42) was coupled with diphenylurea phenylacetic acid (21) to give ester (43).

### Preparation of Compound 7 in Table 1

Using the procedure described for the hydrolysis of ester (22), ester (43) was hydrolysed to give Compound 7.

MS 589.4 (M+H)⁺

¹H NMR (DMSO; 300 MHz) 1.00 (3H, d), 1.60 (2H, m), 1.80 (2H, m), 2.10 (4H, m), 2.20 (3H, s), 2.30 (1H, m), 2.60 (1H, m), 3.70 (1H, m), 3.80 (1H, m), 4.10 (1H, m), 6.85 (2H, m), 7.10 (3H, m), 7.20 (2H, m), 7.30 (1H, m), 7.40 (2H, m), 7.85 (1H, m), 8.55 (1H, s), 9.60 (1H, s), 9.95 (1H, s).

HPLC 11.93 min

### EXAMPLE 8

### Preparation of Nitro Compound (44)

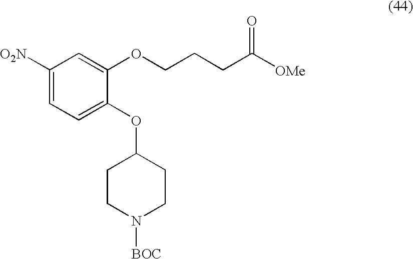

Using the procedure described for preparation of nitro compound (41), nitrophenol (18) was reacted with 1-tert-butoxycarbonyl-4-hydroxypiperidine, triphenylphosphine and diethyl azodicarboxylate to give nitro compound (44).

### Preparation of Aniline (45)

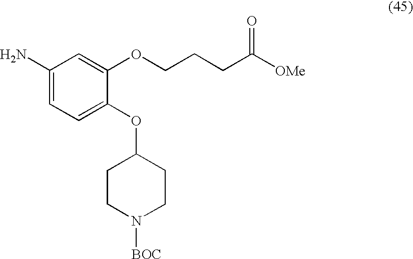

Using the procedure described for preparation of aniline (27), nitro compound (44) was reduced with hydrogen and Pd to give aniline (45).

### Preparation of Ester (46)

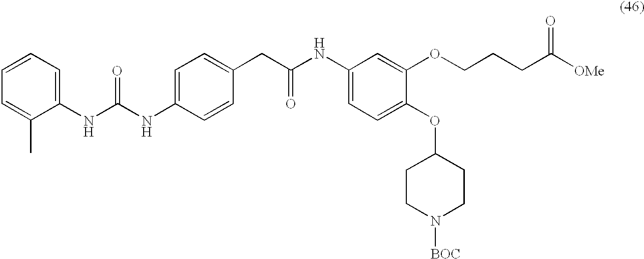

Using the procedure described for preparation of ester (22), aniline (45) was coupled with diphenylurea phenylacetic acid (21) to give ester (46).

### Preparation of Amine (47)

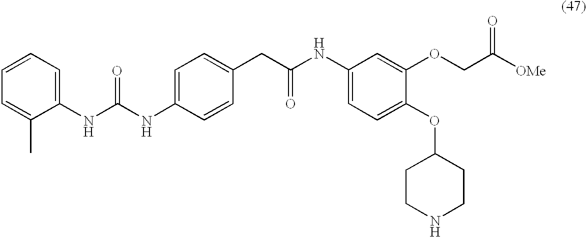

Ester (46) (1.8 g; 2.67 mmol) was dissolved in 90% trifluoroacetic acid (18 cm³) and stirred for 3 h at room temperature. The solvent was removed under reduced pressure and the residue taken up in ethyl acetate and washed with saturated aqueous sodium hydrogen carbonate. This caused a fine precipitate to form giving a colloidal mixture which was filtered. The solid collected was washed with water, ethyl acetate and dried in air to give the product (47) as a colourless solid (1.15 g, 75%). MS 575.2 (M+H)⁺

### Preparation of Ester (48)

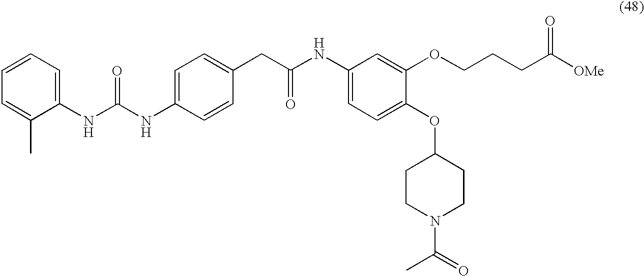

Amine (47) (300 mg; 0.52 mmol) was dissolved in pyridine (5 cm³) and acetic anhydride (59 ml; 0.63 mmol) added. The mixture was stirred at room temperature for 5 h. The solvent was removed under reduced pressure and the residue dissolved in DCM, washed with saturated, aqueous sodium hydrogen carbonate, dried (MgSO4) and concentrated under reduced pressure. The residue was dissolved in MeOH and passed through and Isolute SCX column to give the product (48) as a colourless foam (198 mg, 61%).

¹H NMR (DMSO; 300 MHz) 1.55 (2H, m), 1.80 (2H, m), 1.95 (5H, m), 2.25 (3H, s), 3.30 (2H, m), 3.50 (2H, s), 3.60 (3H, s), 3.70 (2H, m), 3.90 (2H, t), 4.35 (1H, m), 6.90 (2H, d), 7.05-7.15 (3H, m), 7.20 (2H, d), 7.35 (1H, d), 7.40 (2H, d), 7.80 (1H, d), 7.90 (1H, s), 8.98 (1H, s), 9.95 (1H, s).

### Preparation of Compound 8 in Table 1

Using the procedure described for the hydrolysis of ester (22), ester (48) was hydrolysed to give Compound 8.

MS 603.4 (M+H)⁺

¹H NMR (DMSO; 300 MHz) 1.55 (2H, m), 1.80 (2H, m), 1.90 (2H, m), 2.00 (3H, s), 2.25 (3H, s), 2.40 (2H, t), 3.30 (2H, m), 3.50 (2X, s), 3.65 (2H, m), 3.95 (2H, t), 4.35 (1H, m), 6.90 (2H, m), 7.10 (3H, m), 7.20 (2H, d), 7.35 (3H, m), 7.80 (1H, d), 7.90 (1H, s), 9.00 (1H, s), 10.00 (1H, s)

HPLC 12.77 min

### EXAMPLE 9

### Preparation of Ester (49)

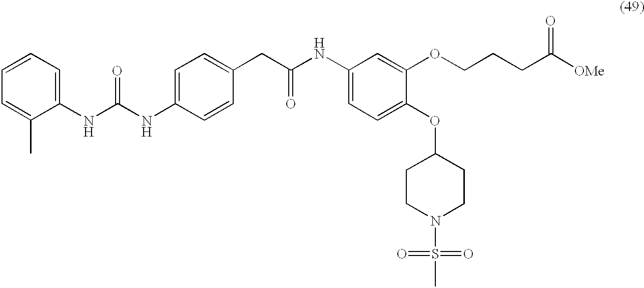

Amine (47) (300 mg; 0.52 mmol) was dissolved in pyridine (5 cm³) and methanesulphonyl chloride (63 ml; 0.63 mmol) was added. The mixture was stirred at room temperature for 5 h. The solvent was removed under reduced pressure and the residue dissolved in DCM, washed with saturated, aqueous sodium hydrogen carbonate, dried (MgSO₄) and concentrated under reduced pressure. The residue was chromatographed (5% MeOH/DCM) to give the product (49) as a yellow oil (112 mg, 33%).

MS 652.5 (M+H)⁺

### Preparation of Compound 9 in Table 1

Using the procedure described for the hydrolysis of ester (22), ester (49) was hydrolysed to give Compound 9.

LC-MS 3.90 639.5 (M+H)⁺

¹H NMR (DMSO; 300 MHz) 1.75 (2H, m), 1.90 (4H, m), 2.20 (3H, s), 2.40 (2H, t), 2.90 (3H, s), 3.10 (2H, m), 3.50 (2H, s), 3.60 (2H, m), 3.90 (2H, t), 4.30 (1H, m), 6.95 (2H, m), 7.10 (3H, m), 7.20 (2H, d), 7.40 (2H, m), 7.80 (1H, d), 7.90 (1H, s), 9.00 (1H, s), 10.00 (1H, s)

### EXAMPLE 10

### Preparation of Nitro Compound (50)

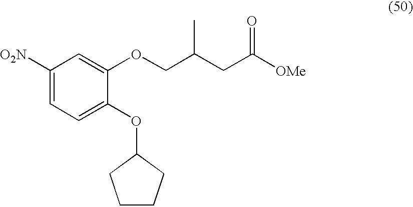

Using the procedure described for nitro compound (41), nitrophenol (30) was reacted with cyclopentanol, triphenylphosphine and diethyl azodicarboxylate to give nitro compound (50).

### Preparation of Aniline (51)

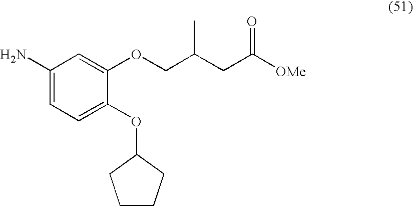

Using the procedure described for preparation of aniline (27), nitro compound (50) was reduced with hydrogen and Pd to give aniline (51).

### Preparation of Ester (52)

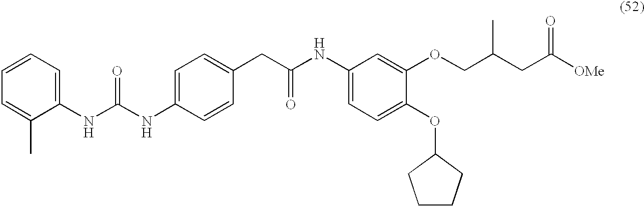

Using the procedure described for preparation of ester (22), aniline (51) was coupled with diphenylurea phenylacetic acid (21) to give ester (52).

### Preparation of Compound 10 in Table 1

Using the procedure described for the hydrolysis of ester (22), ester (52) was hydrolysed to give Compound 10.

LC-MS 3.64 560.5 (M+H)⁺

¹H NMR (DMSO; 300 MHz) 1.00 (3H, d), 1.55 (2H, m), 1.70 (6H, m), 2.15 (1H, dd), 2.25 (4H, m), 2.45 (1H, dd), 3.60 (2H, s), 3.75 (2H, d), 4.70 (1H, m), 6.85 (1H, d), 6.90 (1H, t), 7.05 (1H, dd), 7.10 (2H, m), 7.20 (2H, d), 7.30 (1H, d), 7.40 (2H, d), 7.65 (1H, d), 7.70 (1H, s), 8.95 (1H, s), 9.90 (1H, s), 12.15 (1H, brs)

### EXAMPLE 11

### Preparation of Nitro Compound (53)

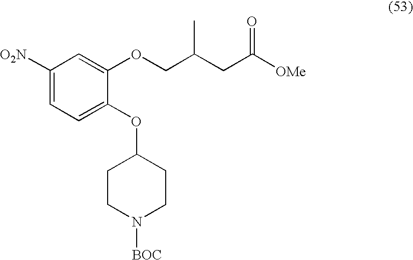

Using the procedure described for preparation of nitro compound (41), nitrophenol (30) was reacted with 1-tert-butoxycarbonyl-4-hydroxypipenidie, triphenylphosphine and diethyl azodicatboxylate to give nitro compbund (53).

### Preparation of Aniline (54)

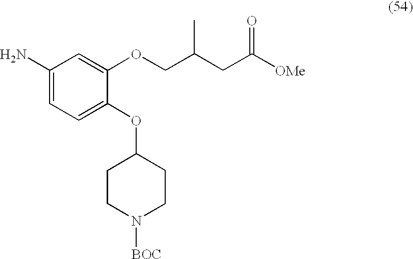

Using the procedure described for preparation of aniline (27), nitro compound (53) was reduced with hydrogen and Pd to give aniline (54).

### Preparation of Ester (55)

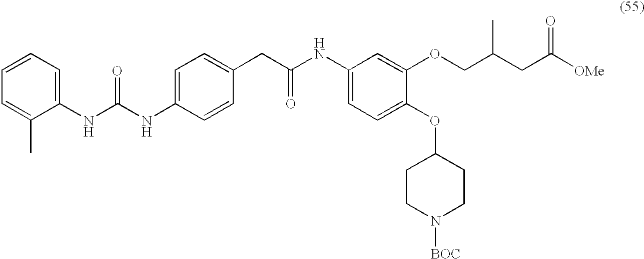

Using the procedure described for preparation of ester (22), aniline (54) was coupled with diphenylurea phenylacetic acid (21) to give ester (55).

### Preparation of Amine (56)

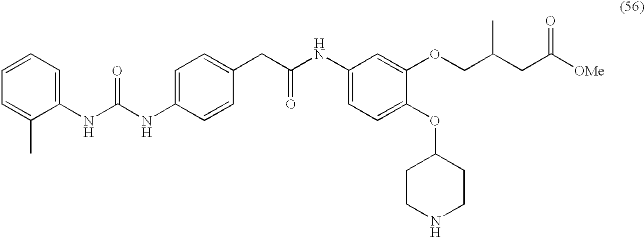

Using the procedure described for preparation of amine (47), ester (55) was treated with trifluoroacetic acid to give amine (56).

### Preparation of Compound 11 in Table 1

Using the procedure described for preparation of ester (49), amine (56) was treated with methanesulphonyl chloride in pyridine. This procedure gave rise to a mixture of products, from which Compound 11 was isolated.

MS 575.7 (M+H)⁺

¹H NMR (DMSO; 300 MHz) 1.00 (3H, d), 1.60 (2H, m), 1.85 (2H, m), 2.10 (1H, dd), 2.20 (3H, s), 2.40 (2H, m), 2.70 (2H, m), 3.00 (2X m), 3.50 (2H, s), 3.80 (2H, m), 4.20 (1H, m), 6.95 (2H, t), 7.00 (2H, m), 7.10 (2H, t), 7.20 (1H, d), 7.40 (3H, m), 7.80 (1H, d), 8.10 (1H, s), 9.20 (1H, s), 10.00 (1H, s).

HPLC 11.65 min

### EXAMPLE 12

### Preparation of Nitro Compound (57)

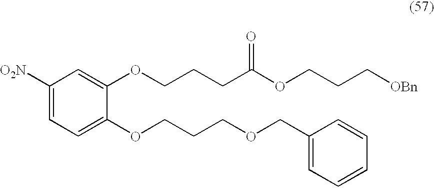

A mixture of the carboxylic acid derivative of nitro phenol (18) (300 mg), 3-benzyloxypropyl bromide (800 mg), DMF (10 mL) and potassium carbonate (334 mg) was stirred at 60° C. for 24 h. The mixture was cooled, treated with water and extracted with EtOAc. The extract was washed with brine, dried and evaporated to dryness under reduced pressure and the residue was purified by flash chromatography using increasingly polar mixtures of EtOAc and hexane as eluant to give (57) as a gum (550 mg).

### Preparation of Ester (58)

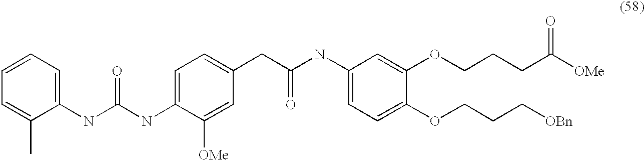

A mixture of nitro compound (57) (540 mg), EtOH (20 ml), 10% palladium on carbon catalyst (200 mg) and ammonium formate (190 mg) was stirred at room temperature for 1 h. The mixture was filtered and the filtrate was evaporated to dryness under reduced pressure and the residue was partitioned between water and EtOAc, and the organic phase was dried and evaporated to dryness. The residue was treated wth DMF (3 ml), 1-hydroxybenzotriazole (200 mg), 1-(3,3-dimethylaminopropyl)-3-ethyl carbodiimide hydrochloride (300 mg), 3-methoxy-4-(2-methylphenylureido)phenylacetic acid (CAS No 181519-21-1, 284 mg) and N-methylmorphoijne (0.2 mL) and the mixture was stirred at room temperature for 24 h. The mixture was treated with EtOAc and washed successively with 1N HCl, 1N NaOH and brine then dried and evaporated to dryness under reduced pressure. The residue was treated with MeOH (30 mL) and concentrated sulphuric acid (0.2 ml) and the mixture was heated at reflux for 3 h. The cooled mixture was diluted with water and extracted with EtOAC, and the extract was washed with brine, dried and evaporated to dryness under reduced pressure. The residue was purified by flash chromatography using increasingly polar mixtures of EtOAC and hexane as eluent to give ester (58) (380 mg).

### Preparation of Compound 12 in Table 1

A mixture of the methyl ester (58) ( 80 mg), DMSO (1 mL) and 2N NaOH (0.2 mL) was stirred at room temperature for 1 h. The mixture was acidified with glacial acetic acid, diluted with water and the gummy precipitate was extracted into a mixture of EtOAc and THF. The extract was washed with brine, dried and evaporated to dryness under reduced pressure. The residue was triturated with a mixture of EtOAc and Et₂O and the insoluble off white solid collected to give Compound 12 (67 mg)

¹H NMR (DMSO; 300 MHz) 1.8-2.0 (4H, m), 2.2 (3H, s), 2.35 (2H, t), 3.5 (2H, s), 3.6 (2H, t), 3.9 (5H, m), 4.0 (2H, t), 4.45 (2H, s), 6.8-6.95 (3H, m), 7.0 (1H, m), 7.05-7.15 (3H, m), 7.2-7.3 (6H, m), 7.75 (1H, d), 8.0 (1H, d), 8.45 (1H, s), 8.55 (1H, s), 9.9 (1H, s).

MS ES− 654(M−H)

### EXAMPLE 13

### Prearation of Methyl Ester (59)

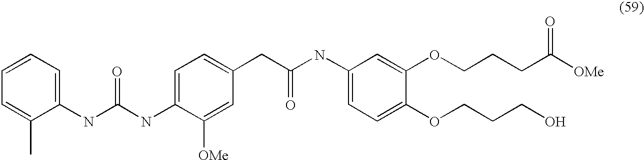

A mixture of the ester (58) (0.28 g), EtOH (25 ml), THF (25 mL) and 10% palladium on carbon catalyst (50 mg) was stirred at room temperature under an atmosphere of hydrogen for 24 h. The mixture was filtered and the filtrate was evaporated to dryness under reduced pressure. The residue was triturated with EtOH and the insoluble solid collected to give ester (59) (205 mg).

### Preparation of Compound 13 in Table 1

A mixture of the methyl ester (59) (50 mg), DMSO (1 mL) and 2N NaOH (0.2 mL) was stirred at room temperature for 1 h. The mixture was acidified with glacial acetic acid, diluted with water and the tan precipitate was collected and washed with water to give Compound 13 (41 mg).

¹ H NMR (DMSO; 300 MHz) 1.8-2.0 (4H, m), 2.2 (3H, s), 2.35 (2H, t), 3.5-3.6 (4H, m), 3.9 (5H, m), 3.95 (2H, t), 4.45 (2H, s), 6.8-6.95 (3H, m), 7.0 (1H, m), 7.05-7.15 (3H, m), 7.3 (1H, m), 7.75 (1H, d), 8.0 (1H, d), 8.45 (1H, s), 8.55 (1H, s), 9.9 (1H, s).

MS ES− 564 (M−H)

### EXAMPLE 14

### Preparation of Ester (60)

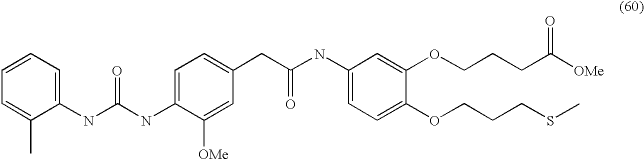

A solution of methanesulphonyl chloride (30 mg) in THF (0.5 mL) was added dropwise to a stirred mixture of the alcohol (59) (140 mg), NMP (2 mL) and triethylamine (0.1 mL) and the mixture was stirred at room temperature for 2 h. The mixture was treated with sodium thiomethoxide (80 mg) and stirred for a further 2 h. The stirred mixture was treated with water (10 mL) and EtOAc (5 mL) and the insoluble solid collected to give ester (60) (95 mg).

### Preparation of Compound 14 in Table 1

A mixture of the methyl ester (60) (85 mg), DMSO (1 mL) and 2N NaOH (0.5 mL) was stirred at room temperature for 1 h. The mixture was acidified with glacial acetic acid, diluted with water and the pale yellow precipitate was collected and washed with water and Et₂O to give Compound 14 (68 mg).

¹H NMR (DMSO; 300 MHz) 1.8-2.0 (4H, m), 2.05 (3H, s), 2.2 (3H, s), 2.4 (2H, t), 2.6 (2H, t), 3.5 (2H, s), 3.85-4.0 (7H, m), 6.8-6.95 (3H, m), 7.0 (1H, m), 7.05-7.15 (3H, m), 7.3 (1H, m), 7.75 (1H, d), 8.0 (1H, d), 8.45 (1H, s), 8.55 (1H, s), 9.9 (1H, s).

MS ES+ 596 (M+H)

### EXAMPLE 15

### Preparation of Amine (61)

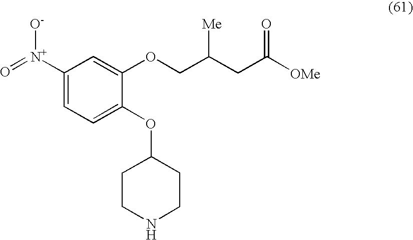

Nitro compound (53) (3.26 g, 7.21 mmol) was dissolved in 4M hydrochloric acid in 1,4-dioxane (10 cm³) and stirred at room temperature for 6 h. The solvent was removed under reduced pressure and the residue dissolved in DCM, washed with saturated aqueous sodium hydrogen carbonate, dried (MgSO₄) and concentrated under reduced pressure to give the product amine (61) as an oil (2.97 g)

MS 353.6 (M+H)⁺

### Preparation of Nitro Compound (62)

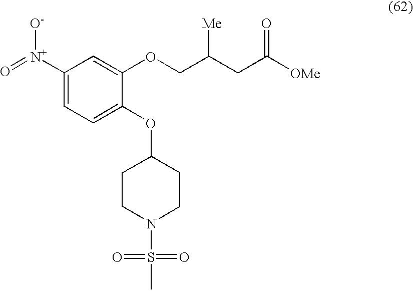

Amine (61) (0.5 g, 1.42 mmol) was dissolved in DCM (5 cm³) and diisopropylethylamine (494 ml; 2.84 mmol) added. The mixture was cooled to 0° C. and methanesulphonyl chloride (165 ml; 2.13 mmol) added. The mixture was stirred at room temperature for 7 h. The mixture was washed with 2N aqueous hydrochloric acid, 2N sodium hydroxide, dried (MgSO₄) and concentrated under reduced pressure. The residue was chromatographed (2% MeOH/DCM) to give the product (62) as an oil (454.8 mg, 74%).

MS 431.5 (M+H)⁺

### Preparation of Aniline (63)

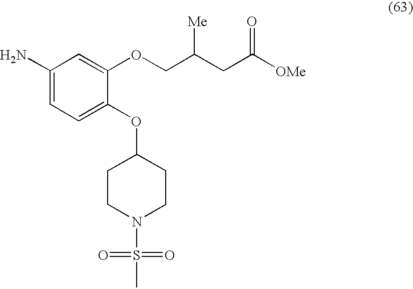

Using the procedure described for preparation of aniline (27), nitro compound (62) was reduced with hydrogen and Pd to give aniline (63).

### Preparation of Ester (64)

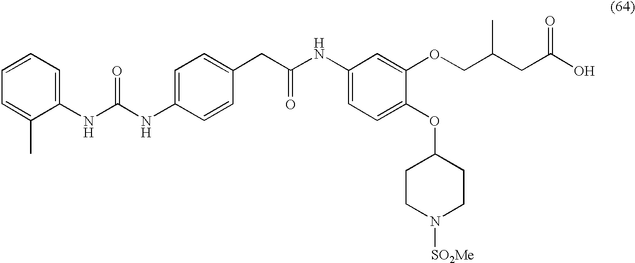

Using the procedure described for preparation of ester (22), aniline (63) was coupled with diphenylurea phenylacetic acid (21) to give ester (64).

### Preparation of Compound 15 in Table 1

Using the procedure described for the hydrolysis of ester (22), ester (64) was hydrolysed to give Compound 15.

MS 653.4 (M+H)⁺

¹H NMR (DMSO; 300 MHz) 1.00 (3H, d), 1.75 (2H, m), 1.85 (2H, m), 2.15 (1H, m), 2.20 (3H, s), 2.25 (1H, m), 2.45 (1H, m), 2.85 (3H, s), 3.10 (2H, m), 3.30 (2H, m), 3.50 (2H, s), 3.80 (2H, d), 4.30 (1H, m), 6.90 (2H, m), 7.10 (3H, m), 7.20 (2H, d), 7.40 (3H, t), 7.90 (1H, d), 8.00 (1H, s), 9.15 (1H, s), 10.00 (1H, s).

### EXAMPLE 16

### Preparation of Nitro Compound (65)

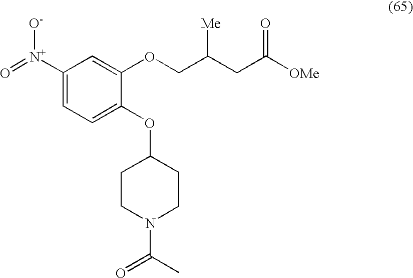

Amine (61) (0.5 g, 1.42 mmol), diisopropylethylamine (494 ml; 2.84 mmol) and 4-dimethylaminopyridine (catalytic) were dissolved in DCM (5 cm³) and cooled to 0° C. Acetic anhydride (201 ml; 2.13 mmol) was added and the mixture stirred at room temperature for 5 h. The mixture was washed with 2N aqueous hydrochloric acid, 2N sodium hydroxide, dried (MgSO₄) and concentrated under reduced pressure. The residue was dissolved in MeOH and washed through an Isolute SCX column to give the product (65) as an oil (475 mg, 85%).

MS 395.6 (M+H)⁺

### Preparation of Aniline (66)

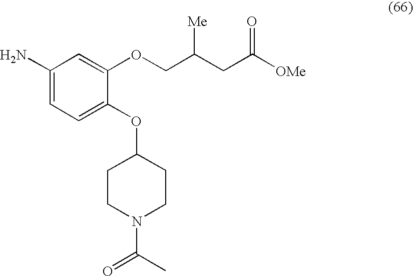

Using the procedure described for preparation of aniline (27), nitro compound (65) was reduced with hydrogen and Pd to give aniline (66).

### Preparation of Ester (67)

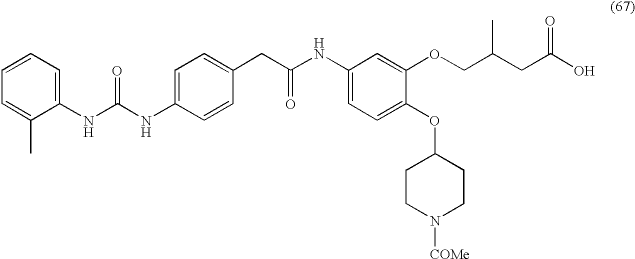

Using the procedure described for preparation of ester (22), aniline (66) was coupled with diphenylurea phenylacetic acid (21) to give ester (67).

### Preparation of Compound 16 in Table 1

Using the procedure described for the hydrolysis of ester (22), ester (67) was hydrolysed to give Compound 16.

MS 617.6 (M+H)⁺

¹H NMR (DMSO; 300 MHz) 1.00 (3H, d), 1.55 (2H, m), 1.80 (2H, m), 1.95 (3H, s), 2.15 (1H, m), 2.20 (4H, m), 2.25 (1H, m), 3.30 (2H, m), 3.50 (2H, s), 3.60 (2H, m), 3.80 (2H, m), 4.30 (1H, m), 6.90 (2H, m), 7.10 (3H, m), 7.20 (2H, d), 7.40 (3H, t), 7.80 (1H, d), 7.90 (1H, s), 9.00 (1H, s), 10.00 (1H, s).

HPLC 13.55 min

### EXAMPLE 17

### Preparation of Compound 17 in Table 1

A mixture of Compound 14 (45 mg), MeOH (1 mL), Oxone (100 mg) and water (0.5 mL) was stirred at room temperature for 72 h;, diluted with water, and the insoluble solid collected. The solid was stirred with a mixture of DMSO (0.5 mL) and 2N NaOH (0.1 mL) at room temperature for 1 h and acidified with glacial acetic acid. The mixture was diluted with water and the lighit brown precipitate collected and washed with water to give Compound 17 (31 mg).

¹H NMR (DMSO, 300 MHz) 1.9 (2H, m), 2.1 (2H, m), 2.2 (3H, s), 2.4 (2H, t), 3.0 (3H, s), 3.25 (2H, m), 3.5 (2H, s), 3.85-4.05 (7H, m), 6.8-6.95 (3H, m), 7.0 (1H, m), 7.05-7.15 (3H, m), 7.3 (1H, m), 7.75 (1H, d), 8.0 (1H, d), 8.45 (1H, s), 8.55 (1H, s), 9.9 (1H, s).

MS ES+ 628 (M+H)

### EXAMPLE 18

The compounds of the invention or pharmaceutically acceptable salts thereof may be formulated into tablets together with, for example, lactose Ph.Eur, Croscarmellose sodium, maize starch paste (5% w/v paste) and magnesium stearate for therapeutic or prophylactic use in humans. The tablets may be prepared by conventional procedures well known in the pharmaceutical art and may be film coated with typical coating materials such as hydroxypropylmethylcellulose.

### In Vitro and In Vivo Assays

The following abbreviations are used. Suitable sources of materials are listed below.

MOLT-4 cells—human T-lymphoblastic leukaemia cells (European Collection of Animal Cell Cultures, Porton Down)

Fibronectin—purified from human plasma by gelatin-sepharose affinity chromatography according to the methods described in E. Nengvall, E. Ruoslahti, Int. J. Cancer, 1977, 20, pages 1-5 and J. Forsyth et al, Methods in Enzymology, 1992, 215, pages 311-316).

RPMI 1640—cell culture medium. (Life technologies, Paisley UK).

PBS—Dulbecco's phosphate buffered saline (Life Technologies).

BSA—Bovine serum albumin, fraction V (ICN, Thame, UK).

CFA—Complete Freund's Adjuvant (Life Technologies).

In the following assays and models references to compound(s) refers to the compounds of formula (I) and (II) according to the present invention.

1.1 In vitro assay

1.1.1 MOLT-4 cell/Fibronectin adhesion assay.

The MOLT-4 cell/fibronectin adhesion assay was used to investigate the interaction of the integrin α₄-β₁expressed on the MOLT-4 cell membrane with fibronectin. Polystyrene 96 well plates were coated overnight at 4° C. with fibronectin, 100 μl of 10 μg/ml in PBS. Non-specific adhesion sites were blocked by adding 100 μl BSA, 20 mg/ml. After incubating for 1 h at room temperature, the solutions were aspirated. MOLT-4 cells suspended in serum-free RPMI-1640 medium 2E6 cells/ml (50 μl) and solutions of compound diluted in the same medium (50 μl) were added to each well. After incubation for 2 h at 37° C. in a humidified atmosphere of 5% (v/v) CO₂, non-adherent cells were removed by gentle shaking followed by vacuum aspiration. Adherent cells were quantified by a colorimetric acid phosphatase assay. To each well was added 100 μl p-nitrophenyl phosphate (6 mg/ml) in 50 mM sodium acetate buffer, pH 5.0, containing 1% Triton X-100. After incubation for 1 h at 37° C., 50 μl sodium hydroxide (1M) was added to each well and the absorbance 405 nm was measured on a microplate spectrophotometer. Compounds which inhibited adhesion gave a lower absorbance reading. Standard, control and test conditions were assayed in triplicate. Percentage inhibition was calculated with respect to total (no inhibitor) and non-specific (no fibronectin) standards on each plate. In this assay, it was found that Compound 1 in Table 1 is an inhibitor at 87 nm and Compound 2 in Table 1 is an inhibitor at 84 nM.

1.2 In-vivo Inflammation Models

Activity of a compound can be tested in the following models.

1.2.1 Ovalbumin Delayed type Hypersensitivity in mice

Balb/c female mice (20-25 g) are immunised on the flank with an 1:1 (v/v) emulsion of ovalbumin (2 mg/ml) with CFA. Seven days later the mice are challenged by subplantar injection of 1% heat aggregated ovalbumin in saline (30 μl) into the right hind foot pad. Swelling of the foot develops over a 24 hour period following which foot pad thickness is measured and compared with the thickness of the contralateral uninjected foot. The percentage increase in foot pad thickness is calculated. Compounds are dosed orally by gavage to groups of 5 mice at doses ranging from 0.001 mg/kg to 100 mg/kg. Inhibition of the inflammatory response is calculated comparing vehicle treated animals and compound treated groups.

1.2.2. Collagen-induced arthritis in mice

DBA/1 male mice are immunised with 0.1 ml of an emulsion prepared from equal volumes of bovine collagen type II in 0.05M acetic acid (2 mg/ml) and CFA. This mixture is injected at the base of the tail. Twenty days later compounds are dosed orally by gavage at doses ranging from 0.001 mg/kg/day to 100 mg/kg/day. On the day following the first dose, each animal receives an intra-peritoneal booster injection of 0.1 ml of collagen type II in acetic acid. The mice are assessed for the incidence and severity of arthritis in all four limbs for up to 28 days. Inhibition of arthritis is calculated by comparing vehicle treated and compound treated mice.

1. A compound of formula (I) 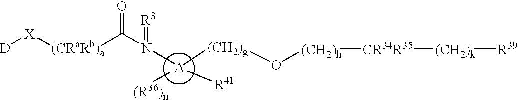
where D is a VLA-4 specificity determinant which does not impart significant IIB/IIIa activity;
R^{a} and R^{b} are independently hydrogen or C₁₋₄ alkyl;
a is an integer from 1 to 4;
X is a direct bond, oxygen, sulphur, amino or C₁₋₄alkylammno;
R³ is hydrogen or C₁₋₅ alkyl;
A is aryl or heterocycle;
n is 0 or an integer of from 1 to 3;
R³⁴ is hydrogen, C₁₋₆ alkyl, aryl or heterocycle, the aryl or heterocycle being optionally substituted with one or more substituents independently selected fiom nitro, C₁₋₆alkyl, C₂₋₆alkenyl, C₂₋₆alkynyl, C₁₋₄alkoxy, C₁₋₆alkylamino, C₁₋₄alkylC₁₋₆alkyoxyl, C₁₋₆alkylaminoC₁₋₆alkyl, cyano, halogeno, trifluoromethyl, hydroxy, (CH₂)ₚOH where p is 1 or 2, —CO₂R^{e1} and —CONR^{e1}R^{f1}, where R^{e1} and R^{f1} are independently selected from hydrogen and C₁₋₆ alkyl;
R³⁵ is selected from hydrogen, hydroxy, C₁₋₆ alkyl, C₂₋₆alkenyl, 1,3-benzodioxol-5-yl, an ester group, amido, heterocycle and aryl, the heterocycle, and aryl optionally substituted with one or more substituents independently selected from nitro, C₁₋₆alkyl, C₂₋₆alkenyl, C₂₋₆alkynyl, C₁₋₄alkoxy, C₁₋₆alkylamino, C₁₋₄alkylC₁₋₆alkyoxyl, C₁₋₆alkylaminoC₁₋₆alkyl, cyano, halogeno, trifluoromethyl, hydroxy, (CH₂)ₚOH where p is 1 or 2, —CO₂R^{e2} and —CONR^{e2}R^{f2}, where R^{e2} and R^{f2} are independently selected from hydrogen and C₁₋₆ alkyl;
each R³⁶ group, which may be the same or different, is independently selected from C₁₋₆alkyl, C₂₋₆alkenyl, C₂₋₆alkynyl, C₁₋₄alkoxy, C₁₋₄alkanoyl, C₁₋₆alkylamino, C₁₋₄alkoxylC₁₋₆alkyl, C₁₋₆alkylaminoC₁₋₆alkyl, nitro, cyano, halogeno, trifluoromethyl, hydroxy, (CH₂)ₚOH where p is 1 or 2, —CO₂R^{e3}, and —CONR^{e3}R^{f3}, where R^{e3} and R^{f3} are independently selected from hydrogen and C₁₋₆ alkyl;
R³⁹ is an acidic functional group;
h is zero or 1;
g is zero of 1;
k is zero or a number from 1 to 3;
and R⁴¹ is a group of formula (V)
U - (CH₂)_{d} - V -T (V)
wherein U is selected from oxygen, sulphur, a direct bond or —CH₂O—, V is selected from nitrogen, oxygen, sulphur, S(O), S(O)₂ or a direct bond, d is zero or a number from 1 to 4, and T is selected from R^{c} or, when V is nitrogen, R^{c}R^{d}, where R^{c} and R^{d} are independently selected from hydrogen, C₁₋₄ alkyl, C₁₋₄ alkoxy, C₁₋₄ alkoxy(C₁₋₆)alkyl, C₃₋₇ cycloalkyl, aralkyl or aryl; or T is a heterocycle containing up to three heteroatoms selected from nitrogen, oxygen and sulphur, optionally substituted with one or more substituents selected from C₁₋₆alkyl, C₂₋₆alkenyl, C₂₋₆alkynyl, C₁₋₆ alkoxy, C₁₋₄alkanoyl, C₁₋₆ alkylamino, C₁₋₄alkoxylC₁₋₆alkyl, C₁₋₆alkylaminoC₁₋₆alkyl, C₁₋₄ alkylsulphonyl, nitro, cyano, halogeno, trifluoromethyl, trifluoromethoxy, hydroxy, (CH₂)ₚOH where p is 1 or 2, —CO₂R^{e4}, and —CONR^{e4}R^{f4}, where R^{e4} and R^{f4} are independently selected from hydrogen and C₁₋₆alkyl, and linked to V through a ring carbon or nitrogen and with the provisos that when T is a heterocycle linked to V through a ring nitrogen then V is a direct bond, and that at least one of U or V is other than a direct bond or d is othr than 0;
or a pharmaceutically acceptable salt or in vivo hydrolysable derivative thereof.

2. A compound according to claim 1 where A is a 5 or 6-membered heterocycle or phenyl.

3. A compound according to claim 1 or claim 2 wherein D is a group of sub-formula (iii) 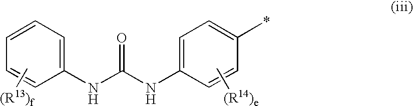
where * is the point of attachment to the group X in formula (I), each R¹³ or R¹⁴ is independently selected from C₁₋₆alkyl, C₂₋₆alkenyl, C₂₋₆alkynyl, C₁₋₄alkoxy, C₁₋₄ alkanoyl, C₁₋₆alkylamino, C₁₋₄alkoxylC₁₋₆alkyl, C₁₋₆alkylaminoC₁₋₆alkyl, cyano, nitro, halogeno, trifluoromethyl, hydroxy, (CH₂)ₚOH where p is 1 or 2, —CO₂R^{e5}, and —CONR^{e5}R^{f5}, where R^{e5} and R^{f5} are independently hydrogen and C₁₋₆alkyl, or two adjacent substituents can be taken together to form a 5-7 membered ring; and
f and e are independently selected from zero or an integer from 1 to 5.

4. A compound according to any one of the preceding claims which is of formula (II) 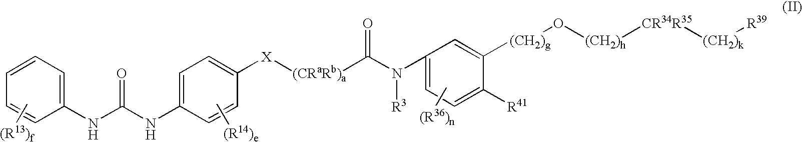
wherein X, R^{a}, R^{b}, a, R³, R³⁶, n, R⁴¹, g, h, R³⁴, R³⁵, k, R³⁹ and R⁴¹ are as defined in claim 1;
each R¹³ or R¹⁴ is independently selected from C₁₋₆alkyl, C₂₋₆alkenyl, C₂₋₆alkynyl, C₁₋₄alkoxy, C₁₋₄alkanoyl, C₁₋₆alkylamino, C₁₋₄alkoxylC₁₋₆alkyl, C₁₋₆alkylaminoC₁₋₆alkyl, cyano, nitro, halogeno, trifluoromethyl, hydroxy, (CH₂)ₚOH where p is 1 or 2, —CO₂R^{e5}, and —CONR^{e5}R^{f5} where R^{e5} and R^{f5} are independently hydrogen and C₁₋₆alkyl, or two adjacent substituents can be taken together to form a 5-7 membered ring;
f and e are independently selected from zero or an integer from 1 to 5.

5. A compound according to claim 4 of formula (III) 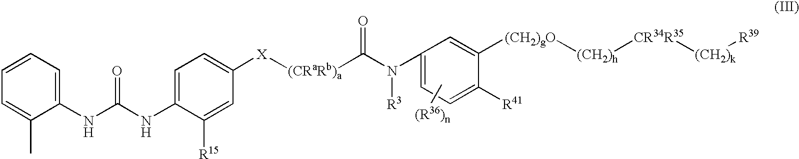
wherein X, R^{a}, R^{b}, a, R³, R³⁶ n, R⁴¹, g, h, R³⁴, R³⁵, k, R³⁹and R⁴¹ are as defined in claim 1; and R¹⁵ is hydrogen or C₁₋₄alkoxy.

6. A pharmaceutical composition which comprises a compound of formulae (I) as defined in claim 1, (II) as defined in claim 4 or (III) as defined in claim 5 or a pharmaceutically acceptable salt or an in vivo hydrolysable derivative thereof and a pharmaceutically acceptable carrier.

7. A compound of formulae (I) as defined in claim 1, (II) as defined in claim 4 or (III) as defined in claim 5 or a pharmaceutically acceptable salt or an in vivo hydrolysable derivative thereof for use in a method of therapeutic treatment of the human or animal body.

8. A method of treating a disease mediated by the interaction between VCAM-1 and/or fibronectin and the integrin receptor α₄β₁ in need of such treatment which comprises administering to said warm-blooded mammals an effective amount of a compound of formulae (I) as defined in claim 1, (II) as defined in claim 4 or (III) as defined in claim 5 or a pharmaceutically acceptable salt or an in vivo hydrolysable derivative thereof.

9. The use of a compound of formulae (I) as defined in claim 1, (II) as defined in claim 4 or (III) as defined in claim 5 or a pharmaceutically acceptable salt or an in vivo hydrolysable derivative thereof in the production of a medicament for use in the treatment of a disease or medical condition mediated by the interaction between fibronectin and/or VCAM-1 and the integrin receptor α₄β₁.

10. A process for preparing a compound of formula (I) as defined in claim 1 or a pharmaceutically acceptable salt or an in vivo hydrolysable derivative thereof; which process which comprises coupling together a compound of formula (VI) 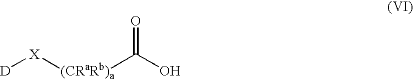
where D, X, R^{a}, R^{b} and a are as defined hereinbefore in relation to formula (I);
and an appropriate amine of formula (VII) 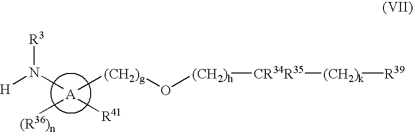
where R³, A, R³⁶, n, R⁴¹, g, h, R³⁴, R³⁵, k and R³⁹ are as hereinbefore defined in relation to formula (I); and
where any functional group is optionally protected;
and thereafter, if necessary:
a) removing any protecting group; and
b) forming a pharmaceutically acceptable salt or in vivo hydrolysable derivative.
